(19) United States
(12) Patent Application Publication
(10) Pub. No.: US 2024/0141129 A1
(43) Pub. Date: May 2, 2024

(54) ENZYMATIC DEGRADATION OF SEMI-CRYSTALLINE POLYMERS AND PLASTIC WASTE CONTAINING SEMI-CRYSTALLINE POLYMERS

(71) Applicants: Hernan Garate, Bures-Sur-Yvette (FR); Ludwik Leibler, Amston, CT (US); Clément Freymond, Issy Les Moulineaux (FR); Andrew Griffiths, Paris (FR); Louise Bréloy, Malakoff (FR); Brian Mansaku, Amston, CT (US); Jack Pallis, Glastonbury, CT (US)

(72) Inventors: Hernan Garate, Bures-Sur-Yvette (FR); Ludwik Leibler, Amston, CT (US); Clément Freymond, Issy Les Moulineaux (FR); Andrew Griffiths, Paris (FR); Louise Bréloy, Malakoff (FR); Brian Mansaku, Amston, CT (US); Jack Pallis, Glastonbury, CT (US)

(21) Appl. No.: 18/480,453

(22) Filed: Oct. 3, 2023

Related U.S. Application Data
(60) Provisional application No. 63/469,767, filed on May 30, 2023.
(60) Provisional application No. 63/412,854, filed on Oct. 3, 2022.

(51) Int. Cl. C08J 11/10; B29B 17/04

(52) U.S. Cl. CPC C08J 11/105; B29B 17/04; C08J 2300/20; C08J 2300/30

(57) ABSTRACT

The present disclosure is related to systems and methods of enzymatic degradation of crystallizable polymers or copolymers and PC/IPM containing crystallizable polymers or copolymers.

### RELATED APPLICATIONS

This application claims priority under 35 U.S.C. § 119(e) to U.S. Provisional Patent Application No. 63/469,767, filed May 30, 2023, and entitled “ENZYMATIC DEGRADATION OF SEMI-CRYSTALLINE POLYMERS AND PLASTIC WASTE CONTAINING SEMI-CRYSTALLINE POLYMERS,” which is incorporated herein by reference in its entirety for all purposes. This application also claims priority under 35 U.S.C. § 119(e) to U.S. Provisional Patent Application No. 63/412,854, filed Oct. 3, 2022, and entitled “ENZYMATIC DEGRADATION OF SEMI-CRYSTALLINE POLYMERS AND PLASTIC WASTE CONTAINING SEMI-CRYSTALLINE POLYMERS,” which is incorporated herein by reference in its entirety for all purposes.

### TECHNICAL FIELD

Enzymatic degradation of crystallizable polymers or copolymers and PC/IPM containing crystallizable polymers or copolymers is generally described.

### SUMMARY

The present disclosure is related to systems and methods of enzymatic degradation of crystallizable polymers or copolymers and PC/IPM containing crystallizable polymers or copolymers. The subject matter of the present invention involves, in some cases, interrelated products, alternative solutions to a particular problem, and/or a plurality of different uses of one or more systems and/or articles.

In some aspects, methods of processing a crystallizable polymer or copolymer and/or a PC/IPM containing the crystallizable polymer or copolymer are provided.

In some embodiments, the method of processing a crystallizable polymer or copolymer and/or a PC/IPM containing the crystallizable polymer or copolymer comprises milling the crystallizable polymer or copolymer to reduce a percentage of crystallinity of the crystallizable polymer or copolymer; and combining the milled crystallizable polymer or copolymer with an enzyme capable of facilitating degradation of the milled crystallizable polymer or copolymer.

In some embodiments, the method of processing a crystallizable polymer or copolymer and/or a PC/IPM containing the crystallizable polymer or copolymer, comprises milling the crystallizable polymer or copolymer to reduce a percentage of crystallinity of the crystallizable polymer or copolymer, wherein a yield of enzymatic degradation after the milling measured after 23 hours of reaction is at least 1.15 times the yield of enzymatic degradation under otherwise essentially identical conditions.

In some embodiments, the method of processing a crystallizable polymer or copolymer and/or a PC/IPM containing the crystallizable polymer or copolymer comprises milling the crystallizable polymer or copolymer to reduce a percentage of crystallinity of the crystallizable polymer or copolymer, wherein a rate of enzymatic degradation per unit equivalent surface area of the crystallizable polymer or copolymer after the milling is at least 1.15 times faster than the rate of enzymatic degradation per unit equivalent surface area of the crystallizable polymer or copolymer prior to the milling, under otherwise essentially identical conditions, with equivalent surface area being calculated as the surface area of a spherical particle having diameter equal to the average particle size prior and after the milling.

In some aspects, methods of processing a post-consumer and/or post-industrial polymeric material are provided.

In some embodiments, the method of processing a post-consumer and/or post-industrial polymeric material comprises milling the post-consumer and/or post-industrial polymeric material to reduce a percentage of crystallinity of the post-consumer and/or post-industrial polymeric material, wherein the post-consumer and/or post-industrial polymeric material comprises a crystallizable polymer or copolymer, and wherein a rate of reduction of the percentage of crystallinity of the post-consumer and/or post-industrial polymeric material comprising the crystallizable polymer or copolymer is at least 1.1 times faster than the rate of reduction of the percentage of crystallinity of a virgin plastic comprising the crystallizable polymer or copolymer, under otherwise identical milling conditions.

In some embodiments, a method of processing a crystallizable polymer or copolymer and/or a PC/IPM containing the crystallizable polymer or copolymer, comprising milling the crystallizable polymer or copolymer to reduce a percentage of crystallinity of the crystallizable polymer or copolymer in a mill, at an apparent mass flow rate of at least 0.01 kg of polymer per hour within the mill, and/or with a mean residence time of the polymer within the mill of less than 30 minutes, at a mill temperature no more than T_{g}+100° C., wherein T_{g} is the glass transition temperature of the crystallizable polymer or copolymer.

In some aspects, compositions are provided.

In some embodiments, the composition comprises a plurality of polymeric particles comprising milled crystallizable polymer or copolymer, wherein the milled crystallizable polymer or copolymer has a percentage of crystallinity that is at least 2% less than the percentage of crystallinity of the crystallizable polymer or copolymer before the milling; and an enzyme capable of facilitating degradation of the plurality of polymeric particles.

In some embodiments, the composition comprises a plurality of polymeric particles comprising milled crystallizable polymer or copolymer, wherein a rate of enzymatic degradation per unit equivalent surface area of the milled crystallizable polymer or copolymer is at least 1.05 times faster than the rate of enzymatic degradation per unit equivalent surface area of the crystallizable polymer or copolymer prior to the milling, under otherwise essentially identical conditions; and an enzyme capable of facilitating degradation of the plurality of polymeric particles.

In some embodiments, the composition, comprises: a plurality of polymeric particles comprising milled crystallizable polymer or copolymer, wherein a yield of enzymatic degradation measured after 23 hours of reaction is at least 1.05 times the yield of enzymatic degradation of the crystallizable polymer or copolymer prior to the milling, under otherwise essentially identical conditions; and an enzyme capable of facilitating degradation of the plurality of polymeric particles.

In some embodiments, the composition comprises a plurality of polymeric particles containing a post-consumer and/or post-industrial polymeric material, wherein the post-consumer and/or post-industrial polymeric material has an average degree of crystallinity of less than or equal to 12%; and an enzyme capable of facilitating degradation of the plurality of polymeric particles.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BACKGROUND

Some enzymes can be used to catalyze degradation of solid polymers and plastics in water and be thus used to recycle plastic waste. However, such methods for polymer and plastic degradation may often have undesirably low efficiency and throughput.

Accordingly, improved methods for processing and degrading polymers and plastics are needed.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows the enzymatic depolymerization activity of milled PET particles, obtained by different milling conditions as described in the Example 1, according to some embodiments.

FIG. 2 shows differential scanning calorimetry scans of milled PET particles, obtained by different milling conditions, according to some embodiments.

FIG. 3 shows a film of PET formed at the inner sieve surface during milling in example 4, which blocks the mill, according to some embodiments.

FIG. 4A shows photographs showing the different teeth-to-sieve gap of the mill in a small gap configuration, according to some embodiments.

FIG. 4B shows photographs showing the different teeth-to-sieve gap of the mill in a large gap configuration, according to some embodiments.

FIG. 5 shows the temperature versus time for the milling conditions described in the Example 5, according to some embodiments.

FIG. 6 shows the temperature versus time for the milling conditions described in the Example 6, according to some embodiments.

FIG. 7 shows the temperature versus time for the milling conditions described in the Example 7, according to some embodiments.

FIG. 8 shows the enzymatic depolymerization activity of milled rPET flakes, obtained by the different milling conditions described in the Example 8, according to some embodiments.

FIG. 9 shows the enzymatic depolymerization activity of milled Evian flakes, obtained by the different milling conditions described in the Example 9, according to some embodiments.

FIG. 10 shows the enzymatic depolymerization activity of milled rPET flakes, obtained by the different milling conditions described in the Example 10, according to some embodiments.

FIG. 11 shows the enzymatic depolymerization activity of milled rPET flakes, obtained by the different preparation and milling conditions described in the Example 18.

FIG. 12 shows the sieve temperature vs. milling time for the conditions described in the Example 19 and the comparative example 20, according to some embodiments.

FIG. 13 shows the sieve temperature vs. milling time for the conditions described in the Example 21, according to some embodiments.

FIG. 14 shows the sieve temperature vs. milling time for the conditions described in the Example 19 and comparative examples 22-24, according to some embodiments.

FIG. 15 shows the enzymatic degradation activity of milled particles described in the Example 15.1 and 17.1, according to some embodiments.

FIG. 16 shows the effect of vacuum on rotor mill temperature, according to some embodiments.

FIG. 17 shows the maximum rotor mill throughput, according to some embodiments.

FIG. 18 shows the effect of RPM on rotor receiving bowl temperature at 390 g/hr throughput, according to some embodiments.

FIG. 19 shows the 0.2 gram pulse testing using different ring sieve distances under vacuum, according to some embodiments.

FIG. 20A shows the apparent Mass Flow versus Pulse Mass for 10 cm diameter ring sieve, according to some embodiments.

FIG. 20B shows the apparent Mass Flow versus Pulse Mass for 11.3 cm diameter ring sieve, according to some embodiments.

FIG. 21A shows the temperature vs. time of steady-state limits of retsch ZM200 Mill with a short ring sieve, according to some embodiments.

FIG. 21B shows the temperature vs. time of steady-state limits of retsch ZM200 Mill with a distance ring sieve, according to some embodiments.

FIG. 22 shows the reactions of short and distance ring sieve milled particles with Novozyme HiC 51032, according to some embodiments.

FIG. 23 shows a comparison of the reaction with Novozyme HiC 51032 between milled extruded rPET and controlled milled rPET, according to some embodiments.

FIG. 24 shows the particle size distribution and crystallinity degree for Example 34, according to some embodiments.

FIG. 25 shows pulse tests for Test 2 on the Hosokawa V-UMP 15 hp, according to some embodiments.

FIG. 26 shows particle size distribution and CD for Example 3.3, according to some embodiments.

FIG. 27 shows the particle size distribution and CD for Example 37, according to some embodiments.

FIG. 28 shows the particle Size Distribution and CD for Example 39, according to some embodiments.

FIG. 29 shows the particle size distribution of rPET milled sample obtained by ball milling as described in the Comparative Example 41 by triplicate, according to some embodiments.

FIG. 30 shows the chemical structure of a PET repeating unit, according to some embodiments.

FIG. 31 shows the absorbance vs. mass concentration calibration curve of TPA in NaOH 0.5 wt. % solution, according to some embodiments.

FIG. 32 shows the reaction yield at 24 h of milled and sieved fractions (150-300) μm prepared as described in Example 42.1, Example 42.2, Example 42.3, and Example 42.4 using LCC ICCG at 65° C.

FIG. 33 shows the percentage depolymerization as a function of reaction time for milled Grade C bales, milled Grade C bales followed by a sink/float operation, and extrusion followed by milling and a sink/float operation, according to some embodiments.

### DETAILED DESCRIPTION

Methods of enzymatic degradation of crystallizable polymers or copolymers and/or post-industrial and/or post-consumer polymeric material (PC/IPM) containing crystallizable polymers or copolymers are generally described. Certain aspects of the present disclosure are directed to the discovery that milling can be used to render crystallizable polymers or copolymers and PC/IPM containing crystallizable polymers or copolymers less recalcitrant to enzyme degradation, not just as a result of a mere size reduction of the crystallizable polymers or copolymers. Inventors herein have discovered that through milling, the crystallizable polymers or copolymers and PC/IPM containing crystallizable polymers or copolymers may become more degradable (i.e., digestible) to enzymes, which may thus lead in faster enzymatic degradation.

In one aspect, without wishing to be bound by any particular theory, it is hypothesized that (additional) decrease of recalcitrance, to enzymatic degradation, of milled crystallizable polymers or copolymers and PC/IPM containing crystallizable polymers or copolymers may, at least in part, be associated with a decrease in the degree of crystallinity of the crystallizable polymers or copolymers during the milling process. Certain embodiments are related to the discovery that the use of crystallizable polymers or copolymers or plastics with a degree of crystallinity reduced by a milling process described herein can allow for more efficient enzymatic degradation of the polymers or plastics. That is, the milled crystallizable polymers or copolymers and/or plastics, by having reduced degree of crystallinity, may be more readily degraded by enzymes and subsequently recycled. Some embodiments are related to the discovery that the method described herein may be particularly useful for processing PC/IPMs. For example, an unexpected discovery is that PC/IPMs comprising crystallizable polymers or copolymers may be more readily degraded by enzymes compared to virgin plastics comprising the same crystallizable polymers or copolymers, under otherwise essentially identical conditions.

It has also been recognized, within the context of the present disclosure, that certain embodiments of the compositions and methods described herein can have a number of advantageous effects including, in certain cases, enhanced enzymatic degradation, continuous operation, simplified polymer and/or plastic processing, compatibility with traditional industrial mills, enhanced degradation of mixed and/or contaminated PC/IPMs, cost reduction and improvement of environmental impact of enzyme assisted degradation (e.g., depolymerization) process, operation at ambient temperatures, elimination of pretreatment steps associated with polymer and/or plastic processing, elimination of cryomilling processes, and an increase in the overall efficiency (e.g., higher processing rate and throughput) of crystallizable polymer or copolymer and/or plastic degradation and recycling. In some embodiments, methods are described. The methods can involve, in some embodiments, milling the crystallizable polymer or copolymer and/or PC/IPM containing the crystallizable polymer or copolymer, such that the crystallizable polymer or copolymer and/or PC/IPM is milled into a form that may be more readily degraded by one or more enzymes. The crystallizable polymer or copolymer may be a single type of crystallizable polymer or copolymer or may be a blend of two or more types of crystallizable polymer or copolymer.

Further advantages of certain embodiments of the composition and methods described herein are described below. In some embodiments, extrusion of PC/IPMs and subsequent cooling is not necessary to obtain high enzymatic degradation rates and/or yields. Accordingly, the environmental and/or ecological impact of such processes may be limited and/or lower than conventional polymeric degradation and/or depolymerization processes. In some embodiments, the methods described herein may advantageously degrade common and/or relatively widely produced PC/IPMs, including but not limited to bottle flakes, food containers, or bioriented films, with improved degradation rates and/or yields. Additionally, mixed PC/IPMs and/or contaminated PC/IPMs may be processed without prior sorting and/or separation processes. Milling of PC/IPMs, in certain embodiments, can be conducted at ambient temperatures thus eliminating the needs for cryomilling and/or other relatively low temperature processing methods thereby reducing the cost, complexity, and overall throughput of degradation processes. In certain embodiments, common industrial mills (e.g. hammer mill, centrifugal mill, pin mill) can be implemented to conduct the methods described herein.

Many post-industrial and post-consumer polymeric materials are crystallizable, e.g., can be semi-crystalline when subjected to certain conditions and/or processes (e.g. temperature, pressure, stress, cooling rates from melt, aging, and/or quenching). These materials may be partially and/or fully amorphous as well, under certain conditions which may be different than the aforementioned conditions. Crystallizable polymers or copolymers can include semi-crystalline polymers or copolymers wherein the semi-crystalline polymers or copolymers comprise at least one or more regions of a crystalline phase. Polymers and/or copolymers that may be considered amorphous can be crystallizable when subjected to the aforementioned conditions and/or processes, and therefore, crystallizable polymers or copolymers may include amorphous polymers or copolymers. Those of ordinary skill in the art understand the meaning of each of these terms. As an example, semi-crystalline materials often exhibit some crystalline behavior, but do not always exhibit such behavior under all conditions. It is to be understood that wherever “crystallizable” is used herein, this can include semi-crystalline materials. It is also to be understood that wherever “semi-crystalline” is used herein, this can include crystallizable materials.

In some embodiments, methods of processing a crystallizable polymer or copolymer and/or a PC/IPM containing the crystallizable polymer or copolymer are provided. The crystallizable polymer or copolymer, in certain embodiments, may be contained within a PC/IPM. Non-limiting examples of PC/IPMs containing crystallizable polymers or copolymers may include a post-consumer and/or post-industrial polymeric material and/or a virgin plastic.

A post-consumer and/or post-industrial polymeric material (PC/IPM) may be or may include a manufacturing or compounding scrap or manufactured objects that were never sold to and/or never used by consumers. Post-consumer and/or post-industrial polymeric materials (post-consumer/industrial polymeric materials; PC/IPMs) have generally been a challenging class of materials to recycle. Typically, PC/IPMs include a myriad of polymeric materials (e.g. polymers and/or polymer-based composites, etc).

PC/IPMs are materials the makeup of which will be clearly understood by those of ordinary skill in the art. In one set of embodiments, PC/IPMs are polymeric materials generated by households, and/or by commercial, institutional, and/or industrial entities in their role as end or intermediate users of products which can no longer be used or is undesirable its intended purpose. A PC/IPM can be a polymer material diverted during the manufacturing or commercial process. For example, such materials can be polymers and/or copolymers that have been formed for a particular use, then identified for a subsequent transformation, process, reaction, or interaction, such as recycling.

A virgin plastic, in some embodiments, is a plastic produced directly by the manufacturer and can be subsequently used to produce a plastic by other compounder(s) or manufacturer(s). A virgin plastic and/or a virgin polymeric material generally refers to a polymeric material that has been produced directly from petrochemical feedstock (e.g., crude oil, natural gas) and has not been previously used or processed (e.g., processed into a consumer or industrial product, used in an industrial process). In some embodiments, a virgin plastic and/or polymeric material can be produced from at least a portion of biomass feedstock. In some embodiments, virgin polymeric materials comprises crystallizable polymers or copolymers in virgin form. A virgin plastic and/or a virgin polymeric material is a material the makeup of which is well understood by those of ordinary skill in the art. A virgin plastic, in certain cases, may comprise some amount (if any) of additives (e.g., catalysts, antioxidants, unreacted monomers, plasticizers, etc.) and comprise crystallizable polymers or copolymers containing some comonomers. The post-consumer and/or post-industrial polymeric material, in certain cases, may comprise some amount of additives (e.g., polymers, small molecules such as but not limited to processing aids, dyes, antioxidants, pigments, fillers, etc.) incorporated into the virgin plastic.

In some embodiments, the post-consumer and/or post-industrial polymeric material comprises a post-consumer and/or post-industrial recycled (PC/IR) plastic, e.g., a post-consumer and/or post-industrial polymeric material that has been used (and may include contaminates, additives or chain modifiers, chain extenders, processing aids, fillers, etc.) and that is subsequently recycled. “PC/IR” is a material or materials the makeup of which will be clearly understood by those of ordinary skill in the art. In typical embodiments, such material or materials are plastic (e.g., polymers) that have been formed for a particular use, such as consumer and/or industrial products or processes, then identified for a subsequent transformation, process, reaction, or interaction, such as recycling.

In some embodiments, PC/IPMs comprise post-consumer and/or post-industrial plastic. Post-consumer and/or post-industrial plastic may comprise at least a portion of plastic, in typical embodiments. Plastics, in this context, can be any of a myriad of materials comprising a polymeric material that can be shaped by flow, molded, or otherwise formed into a structure. “Post-consumer and/or post-industrial plastic” are materials the makeup of which will be clearly understood by those of ordinary skill in the art.

In some embodiments, PC/IPMs comprise plastic waste or mixed plastic waste comprising crystalline polymers or copolymers, amorphous polymers or copolymers, and/or crystallizable polymers or copolymers. Plastic waste, in certain embodiments, may comprise any of myriad of materials that are in whole or in part a polymeric material that an owner and/or holder discards, intends to discard, or is required to discard. In certain embodiments, PC/IPMs comprise at least a portion of plastic waste. “Plastic waste” is a material the makeup of which will be clearly understood by those of ordinary skill in the art. It is to be understood that wherever “PC/IPM” is used herein, this can include plastic waste. It is also to be understood that wherever “plastic waste” is used herein, this can include PC/IPM.

In some embodiments, PC/IPMs comprise post-industrial and/or post-consumer plastic waste. In some embodiments, post-industrial and/or post-consumer plastic waste can be a material or mixture in a recycling stream, plastic waste, and/or mixed plastic waste. Post-industrial and/or post-consumer plastic waste, in some cases, can be formed by mechanical and/or chemical processing (e.g., grinding, washing, drying, etc.) raw waste from one or more consumer products, industrial products, and/or industrial processes. “Post-industrial and/or post-consumer plastic waste” is a material the makeup of which will be clearly understood by those of ordinary skill in the art. It is to be understood that wherever “post-industrial and/or post-consumer plastic waste” is used herein, this can include plastic waste. The crystallizable polymer or copolymer may have any of a variety of appropriate percentages of crystallinity. In some embodiments, the crystallizable polymer or copolymer may have a percentage of crystallinity (i.e., degree of crystallinity) of greater than or equal to 1%, greater than or equal to 5%, greater than or equal to 10%, greater than or equal to 25%, greater than or equal to 50%, greater than or equal to 65%, greater than or equal to 75%, greater than or equal to 90%, or more and/or less than or equal to 99%, less than or equal to 90%, less than or equal to 75%, less than or equal to 65%, less than or equal to 50%, less than or equal to 25%, less than or equal to 10%, less than or equal to 5%, or less. Any of the above-referenced ranges are possible (e.g., greater than or equal to 1% and no less than or equal to 90%, greater than or equal to 5% and no less than or equal to 75%, or greater than or equal to 10% and no less than or equal to 65%, etc.). Other ranges are also possible.

The crystallizable polymer or copolymer may include any of a variety of chemistries. In some embodiments, the PC/IPM (e.g., post-consumer and/or post-industrial plastic and/or virgin plastic) comprises one or more crystallizable polymers or copolymers. Non-limiting examples of crystallizable polymers or copolymers may include, but not limited to, polyesters, polyamides, polyolefins, polystyrenes (e.g., syndiotactic polystyrenes), fluoropolymers, polyurethanes, polyether ether ketones, block copolymers comprising crystallizable domains and components, substituted forms of the foregoing, and combinations thereof. Specific non-limiting examples of polyesters include polyethylene terephthalate (PET), poly(lactic acid) (PLA), poly(L-lactic acid) (PLLA), poly(D-lactic acid) (PDLA), polybutylenesuccinate (PBS), polycaprolactone (PCL), poly(ethylene adipate), polybutylene terephthalate (PBT), and combinations thereof. Specific non-limiting examples of polyamides include polyamide 6, poly(beta-caprolactam), polycaproamide, polyamide-6,6, poly(hexamethylene adipamide) (PA6,6), poly(11-aminoundecanoamide) (PA11), polydodecanolactam (PA12), poly(tetramethylene adipamide) (PA4,6), poly(pentamethylene sebacamide) (PA6,10), poly(hexamethylene dodecanoamide) (PA6,12), poly(m-xylyleneadipamide) (PAMXD6), polyhexamethylene adipamide/polyhexamethylene terephthalamide copolymer (PA66/6T), polyhexamethylene adipamide/polyhexamethylene isophthalamide copolymer (PA66/6I), and combinations thereof. Specific non-limiting examples of polyolefins include polyethylene (e.g., high-density polyethylene, low-density polyethylene, etc.), polypropylene, isotactic polypropylene, and combinations thereof. Specific non-limiting examples of fluoropolymers include polytetrafluoroethylene (PTFE), polyvinylidenefluoride (PVDF), and combinations thereof. In some embodiments, the crystallizable polymer or copolymer is a heterogeneous crystallizable polymer or copolymer containing a mixture of polymers having the one or more of the above-referenced chemistries.

In some embodiments, the method comprises milling the crystallizable polymer or copolymer to produce a plurality of polymeric particles. In embodiments in which the crystallizable polymer or copolymer is contained with a crystallizable PC/IPM, milling the crystallizable polymer or copolymer comprises milling the PC/IPM containing the crystallizable polymer or copolymer.

In one set of embodiments, oriented materials, e.g., PC/IPMs and/or other polymers and/or copolymers, are milled and/or otherwise processed in accordance with this disclosure. Aspects of this disclosure can be particularly effective in improving degradation of oriented materials. Polymeric materials can be oriented mono axially or biaxially intentionally and/or through processing conditions such as molding, especially blow molding or other forming techniques. The degree of orientation of a polymer can be measured easily and routinely by those of ordinary skill in the art, for example specifically via Fourier transfer infrared spectroscopy (FT-IR) and/or X-ray diffractometer measurements (XRD). In one set of embodiments, materials processed according to this disclosure can have a degree of orientation, prior to milling, of at least 5%, 10%, 20%, 30%, or higher. One technique for measuring orientation is the Hermans Orientation Function, as is known. Other techniques include birefringent measurements.

The milled crystallizable polymer or copolymer and/or plastic, in some embodiments, may be in the form of a plurality of polymeric particles having any of a variety of appropriate largest cross-sectional dimensions. In some embodiments, at least 50 wt % (e.g., or at least 60 wt %, at least 70 wt %, at least 80 wt %, at least 90 wt %, or 100 wt %) of the plurality of polymeric particles have largest cross-sectional dimensions of greater than or equal to 5 μm, greater than or equal to 10 μm, greater than or equal to 50 μm, greater than or equal to 100 μm, greater than or equal to 250 μm, greater than or equal to 500 μm, greater than or equal to 700 μm, greater than or equal to 1 mm, or greater than or equal to 2.5 mm. In some embodiments, at least 50 wt % (e.g., at least 60 wt %, at least 70 wt %, at least 80 wt %, at least 90 wt %, or all) of the plurality of polymeric particles have largest cross-sectional dimensions of less than or equal to 5 mm, less than or equal to 2.5 mm, less than or equal to 1 mm, less than or equal to 700 μm, less than or equal to 500 μm, less than or equal to 250 μm, less than or equal to 100 μm, less than or equal to 50 μm, or less than or equal to 10 μm. Combination of the above-referenced ranges are possible (e.g., greater than or equal to 5 μm and less than or equal to 5 mm, or greater than or equal to 10 μm and less than or equal to 1 mm, or greater than or equal to 50 μm and less than or equal to 700 μm). Other ranges are also possible.

The milled crystallizable polymer or copolymer and/or plastic, in some embodiments, may be in the form of a plurality of polymeric particles having any of a variety of appropriate average sizes (e.g., maximum cross-sectional dimension or maximum diameter averaged across a set of particles). In some embodiments, the plurality of polymeric particles has an average size of greater than or equal to 5 μm, greater than or equal to 10 μm, greater than or equal to 50 μm, greater than or equal to 100 μm, greater than or equal to 200 μm, greater than or equal to 250 μm, greater than or equal to 500 μm, greater than or equal to 700 μm, greater than or equal to 1 mm, or greater than or equal to 2.5 mm. In some embodiments, the plurality of polymeric particles has an average size of less than or equal to 5 mm, less than or equal to 2.5 mm, less than or equal to 1 mm, less than or equal to 700 μm, less than or equal to 500 μm, less than or equal to 250 μm, less than or equal to 200 μm, less than or equal to 100 μm, less than or equal to 50 μm, or less than or equal to 10 μm. Combination of the above-referenced ranges are possible (e.g., greater than or equal to 5 μm and less than or equal to 5 mm, or greater than or equal to 150 μm and less than or equal to 100 mm, or greater than or equal to 50 μm and less than or equal to 700 μm). Other ranges are also possible.

Particle size and/or surface area per unit volume and/or mass can be controlled or set between comparative processes or comparative tests, and/or measured, by routine techniques well known to those of ordinary skill in the art. In one such technique, equivalence or non-equivalence of average (or threshold) of particle size can be measured and/or controlled by sieving processes known in the art. Different sieves can pass different threshold particle sizes. I.e., an individual sieve can be set to pass particles having a maximum dimension less than a desired value. Multiple sieves can be used, if desired, to separate a set of particles that have only a size above, or below, that threshold size based on whether the particles pass, or do not pass, through a particular sieve. Then, that set of particles can further be isolated by a second, different sieve process. Through successive sieving, a set of particles can be isolated so as to have both a maximum, and minimum dimension within any range desired. As another specific example, spherical or nearly spherical particles (or particles of other shapes) can be isolated having an average diameter or particle cross section equal to the average of the opening size (mesh size) of the sieves used to select the upper and lower cut-offs of the particle sizes.

The milled crystallizable polymer or copolymer and/or plastic (e.g., in the form of a plurality of polymeric particles) may have any of a variety of appropriate average percentage of crystallinity (i.e., degree of crystallinity) values. The milled crystallizable polymer or copolymer and/or plastic may have an average percentage of crystallinity of greater than 0%, greater than or equal to 1%, greater than or equal to 2%, greater than or equal to 5%, greater than or equal to 10%, greater than or equal to 12%, greater than or equal to 15%, greater than or equal to 20%, greater than or equal to 25%, greater than or equal to 30%, greater than or equal to 40%, greater than or equal to 50%, greater than or equal to 75%, or more, and/or less than or equal to 90%, less than or equal to 75%, less than or equal to 50%, less than or equal to 40%, less than or equal to 30%, less than or equal to 25%, less than or equal to 20%, less than or equal to 15%, less than or equal to 12%, less than or equal to 10%, less than or equal to 5%, less than or equal to 2%, less than or equal to 1%, or less. Any of the above-referenced ranges are possible (e.g., greater than or equal to 1% and less than or equal to 90%, greater than or equal to 1% and less than or equal to 50%, greater than or equal to 1% and less than or equal to 15%). Other ranges are also possible. In some embodiments, the milled crystallizable polymer or copolymer is amorphous, such as having a percentage of crystallinity of essentially 0%. The amount of crystallinity, in this regard, is the number of molecules of a polymer that are involved in a crystalline region, as opposed to an amorphous region.

In some embodiments, at least 50 wt % (e.g., or at least 60 wt %, at least 70 wt %, at least 80 wt %, at least 90 wt %, or 100 wt %) of the milled crystallizable polymer or copolymer (e.g., in the form of a plurality of polymeric particles) may have a percentage of crystallinity of greater than 0%, greater than or equal to 1%, greater than or equal to 2%, greater than or equal to 4%, greater than or equal to 10%, greater than or equal to 12%, greater than or equal to 15%, greater than or equal to 20%, greater than or equal to 25%, greater than or equal to 30%, greater than or equal to 40%, greater than or equal to 50%, greater than or equal to 75%, or more, and/or less than or equal to 90%, less than or equal to 75%, less than or equal to 50%, less than or equal to 40%, less than or equal to 30%, less than or equal to 25%, less than or equal to 20%, less than or equal to 15%, less than or equal to 12%, less than or equal to 10%, less than or equal to 5%, less than or equal to 2%, or less than or equal to 1%, or less. Any of the above-referenced ranges are possible (e.g., greater than or equal to 1% and less than or equal to 90%, greater than or equal to 1% and less than or equal to 50%, or greater than or equal to 1% and less than or equal to 40%). Other ranges are also possible. In some embodiments, at least 50 wt % (e.g., or at least 60 wt %, at least 70 wt %, at least 80 wt %, at least 90 wt %, or 100 wt %) of the milled crystallizable polymer or copolymer may be amorphous, such as having a percentage of crystallinity of 0%.

The percentage of crystallinity of a material (e.g., crystallizable polymer or copolymer and/or PC/IPM, the plurality of polymeric particles, etc.) may be determined by performing differential scanning calorimetry according to standard TA-123. The differential scanning calorimetry measurement may be performed on the material by first equilibrating temperature from room temperature to 0° C., followed by maintaining the temperature at 0° C. for 1 minute. The material may be next heated to 300° C. at 10° C./minute to produce a normalized heat flow curve measured as a function of temperature. The percentage of crystallinity (CD) of the material of interest can be calculated from the heat flow and temperature curve by using Equation 1 below:

$$CD = \frac{( Δ {H}_{melt} - Δ {H}_{cold crystallization} )}{Δ {H}_{melt}^{°}} * 100 \qquad ( Equation 1 )$$

where ΔHₘₑₗₜ is the normalized enthalpy of melting of the crystallizable polymer or copolymer and/or PC/IPM, ΔH_{cold crystallization} is the normalized enthalpy of cold crystallization of crystallizable polymer or copolymer and/or PC/IPM, and ΔH°ₘₑₗₜ is the normalized enthalpy of melting of a 100% crystalline polymer and/or PC/IPM at the melting temperature. As an example, ΔH°ₘₑₗₜ for PET is about 140 J/g. The ΔHₘₑₗₜ and ΔH_{cold crystallization} can be determined from the heat flow curve by integrating the area under the melting endothermic peak and the cold crystallization exothermic peak, respectively, e.g., by using a suitable software (e.g., TRIOS). General protocols for determining the degree of crystallinity using TA-123 standard are described in more detail in “Thermal Analysis Application Brief Determination of Polymer Crystallinity by DSC,” by TA Instruments, Inc.

According to some embodiments, milling the crystallizable polymer or copolymer comprises reducing a percentage of crystallinity of the crystallizable polymer or copolymer. In some embodiments, the milled crystallizable polymer or copolymer (e.g., in the form of the plurality of polymeric particles) may have a crystallinity that is at least 1%, at least 2%, at least 3%, at least 5%, at least 10%, at least 20%, at least 30%, at least 40%, at least 50%, or more, and/or up to 60%, up to 70%, up to 80%, up to 90%, up to 95%, up to 97%, or up to 99% less than the crystallinity of the crystallizable polymer or copolymer before milling. Any of the above-referenced ranges are possible (e.g., at least 1% and up to 99%, at least and up to 99%, at least 10% and up to 99%, or at least 30% and up to 99%, etc.). Other ranges are also possible.

The crystallizable polymer or copolymer (e.g., milled or un-milled) may have any of a variety of appropriate glass transition temperatures (T_{g}). In some embodiments, the crystallizable polymer or copolymer may have a glass transition temperature (T_{g}) of greater than or equal to −150° C., greater than or equal to −130° C., greater than or equal to −120° C., greater than or equal to −100° C., greater than or equal to −50° C., greater than or equal to 0° C., greater than or equal to 50° C., greater than or equal to 100° C., greater than or equal to 150° C., greater than or equal to 200° C., greater than or equal to 220° C., or more, and/or less than or equal to 250° C., less than or equal to 200° C., less than or equal to 150° C., less than or equal to 100° C., less than or equal to 50° C., less than or equal to 0° C., less than or equal to −50° C., less than or equal to −100° C., less than or equal to −120° C., or less than or equal to −130° C. Combinations of the above-referenced ranges are possible (e.g., greater than or equal to −150° C. and less than or equal to 250° C., greater than or equal to −130° C. and less than or equal to 220° C., greater than or equal to −120° C. and less than or equal to 200° C.). Other ranges are also possible.

The glass transition temperature of the crystallizable polymer or copolymer may be measured using differential scanning calorimetry using standard TA-309. The crystallizable polymer or copolymer may be heated at a rate of 10° C./minute. General protocols for determining the degree of crystallinity using TA-309 standard are described in more detail in “Measuring the Glass Transition of Amorphous Engineering Thermoplastics,” by TA Instruments, Inc,

Any of a variety of suitable mills may be employed, including, but not limited to a ball mill, a media mill, a centrifugal mill, a planetary mill, a long gap mill, an air classification mill, a pin mill, an attrition mill, a hammer mill, and/or a granulator.

In some embodiments, the milling may be operated under one or more conditions (e.g., temperatures, feed rate, pressure, power, cooling system, etc.) that allow for effective processing of the crystallizable polymer or copolymer and/or plastic, such as allowing for the production of a plurality of polymeric particles having a percentage of crystallinity and/or an average size in one or more ranges described herein.

In some embodiments, the mill may be operated at any of a variety of appropriate milling powers. In some embodiments, a milling powder of at least 0.01 hp, at least 0.1 hp, at least 0.5 hp, at least 0.8 hp, at least 1 hp, at least 10 hp, at least 50 hp, at least 100 hp, at least 500 hp, at least 1000 hp, and/or up to 1500 hp, up to 2000 hp, up to 2500 hp, up to 3000 hp, or more. Combinations of the above-referenced ranges are possible (e.g., at least 0.01 hp and up to 3000 hp, at least 0.5 hp and up to 2000 hp, or at least 0.8 hp and up to 1500 hp). Other ranges are also possible.

In some embodiments, the milling may occur at any of a variety of appropriate maximum temperatures (within the mill). In some embodiments, the milling occurs at a maximum temperature of greater than or equal to 0° C., greater than or equal to 15° C., greater than or equal to 20° C., greater than or equal to 25° C., greater than or equal to 50° C., greater than or equal to 75° C., greater than or equal to 100° C., greater than or equal to 120° C., greater than or equal to 150° C., or greater than or equal to 200° C., or more, and/or less than or equal to 250° C., less than or equal to 200° C., less than or equal to 150° C., less than or equal to 120° C., less than or equal to 100° C., less than or equal to 75° C., less than or equal to 50° C., less than or equal to 25° C., less than or equal to 20° C., or less than or equal to 15° C. Combinations of the above-referenced ranges are possible (e.g., greater than or equal to 0° C. and less than or equal to 250° C., greater than or equal to 15° C. and less than or equal to 250° C., or greater than or equal to 20° C. and less than or equal to 250° C.). Other ranges are also possible.

In some embodiments, during milling, the rate of temperature increase within the mill may be greater than or equal to 0.1° C. per minute, greater than or equal to 0.25° C. per minute, greater than or equal to 0.5° C. per minute, greater than or equal to 1° C. per minute, greater than or equal to 2° C. per minute, greater than or equal to 5° C. per minute, greater than or equal to 10° C. per minute, greater than or equal to 15° C. per minute, greater than or equal to 25° C. per minute, or more, and/or less than or equal to 50° C. per minute, less than or equal to 25° C. per minute, less than or equal to 15° C. per minute, less than or equal to 12° C. per minute, less than or equal to 10° C. per minute, less than or equal to 5° C. per minute, less than or equal to 1° C. per minute, less than or equal to 0.5° C. per minute, less than or equal to 0.25° C. per minute, or less. Combination of the above-referenced ranges are possible (e.g., greater than or equal to 0.1° C. per minute and less than or equal to 50° C. per minute). Other ranges are also possible.

The one or more temperatures described above within the mill may be determined by measuring the temperature at any suitable stationary part within the mill using a temperature sensor and/or probe. For example, according to some embodiments, the temperature within the mill may be determined by measuring the temperature of a location on a sieve within the mill and/or temperature of air in different locations within the mill.

During milling, the crystallizable polymer or copolymer and/or the PC/IPM containing crystallizable polymer or copolymer may be fed into the mill at a variety of appropriate feed rates. In some embodiments, the crystallizable polymer or copolymer or the PC/IPM containing crystallizable polymer or copolymer may be fed into a mill at a rate of greater than or equal to 0.2 grams, greater than or equal to 0.5 grams per minute, greater than or equal to 1 gram per minute, greater than or equal to 10 grams per minute, greater than or equal to 100 grams per minute, greater than or equal to 1 kilogram per minute, or greater than or equal to 10 kilograms, and/or less than or equal to 50 kilograms per minute, less than or equal to 10 kilograms, less than or equal to 1 kilogram per minute, less than or equal to 100 grams per minute, less than or equal to 10 grams per minute, less than or equal to 1 gram per minute, or less than or equal to 0.5 grams per minute. Combination of the above-referenced ranges are possible (e.g., 0.2 grams per minute and less than or equal to 50 kilograms per minute). Other ranges are also possible.

In some embodiments, a vacuum (e.g., in the form of a vacuum powered cyclone) may be applied to the mill during operation to drive air circulation during milling. For example, in some embodiments, a rotating air stream may be generated inside the cyclone either by a vacuum connected to an upper outlet of the cyclone and/or by the rotor revolutions on the mill to which it is attached. By generating a rotating air stream, heat may be dissipated from the mill as desired, which may allow for adequate cooling within the mill and a higher throughput milling process. In some embodiment, the mill may be operated (substantially) continuously.

In some embodiments, the method comprises exposing and/or combining at least a portion (e.g., some or all) of the milled crystallizable polymer or copolymer with an enzyme capable of facilitating degradation of the milled crystallizable polymer or copolymer. The enzyme, in certain embodiments, may be capable of facilitating depolymerizing of the milled crystallizable polymer or copolymer into smaller units, such as in the form of its base unit (e.g., monomers). In some embodiments, the enzyme may be an enzyme capable of facilitating hydrolysis of the milled crystallizable polymer or copolymer in the presence of water. In some embodiments, the method comprises exposing and/or combining at least a portion (e.g., some or all) of the milled crystallizable polymer or copolymer (in the form of a plurality of polymeric particles) with an enzyme in the presence of water or an aqueous solution.

In some embodiments, the method comprises further degrading (e.g., depolymerizing) at least a portion (some or all) of the milled crystallizable polymers or copolymers with the enzyme. In some embodiments, the milled crystallizable polymer or copolymer may be exposed and/or combined to the enzyme under one or more conditions (e.g., pH, temperature, etc.) suitable for enzymatic degradation. For example, the exposing and/or combining may occur in an environment having a pH of between 1 and 14, between 4 and 12, or between 6 and 11. The pH may be modulated in any of a variety of manners, such as via the addition of base (at desired intervals during the enzymatic degradation process), a buffer having a particular buffer concentration, etc. Non-limiting examples of a buffer may include sodium phosphate, potassium phosphate, glycine buffer, Tris-HCl, etc. In some embodiments, the milled crystallizable polymer or copolymer may be combined to (and degraded by) the enzyme at any of a variety of suitable temperatures, such as between 0° C. and 150° C., between 40° C. and 130° C., or between 60° C. and 120° C.

Any of a variety of appropriate enzymes may be employed. Non-limiting examples of enzymes capable of facilitating degradation (e.g., depolymerization) of the milled crystallizable polymer or copolymer include one or more of hydrolase, esterase, protease, serine protease, metalloprotease, cutinase, lipase, oxidase, peroxidase, and amidase. In one set of embodiments, the enzyme comprises genetically modified enzyme LCC-ICCG. In one set of embodiments, the enzyme comprises commercially available Novozym 51032 (CAS 9001-62-1).

In some embodiments, the milled crystallizable polymer or copolymer may have a relatively higher rate of enzyme degradation compared to the crystallizable polymer or copolymer prior to (or without) the milling, under otherwise essentially identical conditions. Without wishing to be bound by any particular theory, it is believed that by reducing the crystallinity of the crystallizable polymers or copolymers, the crystallizable polymer or copolymer may be more readily degraded and digested by enzymes. In some embodiments, a rate of enzymatic degradation per unit equivalent surface area of the crystallizable polymer or copolymer after the milling is at least 1.05 times, at least 1.1, at least 1.15, at least 1.5 times, at least 2 times, at least 3 times, at least 5 times, at least 10 times, at least 25 times, at least 50 times, at least 75 times, or more, and/or up to 100 times, up to 250 times, up to 500 times, or up to 1000 times, faster (or higher) than the rate of enzymatic degradation per unit equivalent surface area of the crystallizable polymer or copolymer prior to (or without) the milling, under otherwise essentially identical conditions. Equivalent surface area is calculated as the surface area of a spherical particle having diameter equal to the (measured) average particle size of the corresponding crystallizable polymer or copolymer prior to or after the milling. Combination of the above-referenced ranges are possible (e.g., at least 1.05 and less than or equal to 1000, at least 1.5 and less than or equal to 500, or at least 2 and less than or equal to 250). Other ranges are also possible. The rate of enzymatic degradation per unit surface area, in some embodiments, refers to the rate of enzymatic degradation per unit of surface area of the crystallizable polymer or copolymer that is accessible to the enzyme. In some embodiments, prior to performing enzymatic degradation, both the crystallizable polymer or copolymer after the milling and the crystallizable polymer or copolymer prior to the milling may be passed through a sieve, such that the polymers tested for enzymatic degradation have comparable sizes. For example, the crystallizable polymer or copolymer after the milling and the crystallizable polymer or copolymer prior to the milling may be sieved such that their average sizes are within less than 20%, less than 10%, or less than 5% of each other.

According to some embodiments, by employing the milling described herein (e.g., employing the milling conditions and/or mills having certain components or properties described herein), milled crystallizable polymer or copolymer with a reduced degree of crystallinity may be produced. However, it should be noted that not all milling methods result in milled crystallizable polymer or copolymer having reduced crystallinity, and that in certain embodiments, other milling methods may be employed to produce milled crystallizable polymers or copolymers with little to no reduction in the degree of crystallinity. For example, in certain embodiments, milling of a crystallizable polymer or copolymer may result in a mere reduction in the size of the crystallizable polymer or copolymer, without resulting in any (or resulting in little to no) reduction in the degree of crystallinity. In accordance with some embodiments, milled crystallizable polymer or copolymer (produced using the milling process described herein) exhibiting a reduction in degree of crystallinity may have a rate of enzymatic degradation (per unit equivalent surface area) that is higher than the rate of enzymatic degradation (per unit equivalent surface area) of milled crystallizable polymer or copolymer (produced from other milling methods) that exhibits little to no reduction in the degree of crystallinity after milling and is otherwise identical (e.g., having identical sizes, polymer type, etc.). In some such embodiments, the milled crystallizable polymer or copolymer exhibiting a reduction in degree of crystallinity after the milling (using methods described herein) may have a rate of enzymatic degradation (per unit equivalent surface area) that is at least 1.1 times, at least 1.15, 1.5 times, at least 2 times, at least 3 times, at least 5 times, at least 10 times, at least 25 times, at least 50 times, at least 75 times, or more, and/or up to 100 times, up to 250 times, up to 500 times, or up to 1000 times, higher than the rate of enzymatic degradation (per unit equivalent surface area) of are otherwise essentially identical milled crystallizable polymer or copolymer (e.g., such as have comparable sizes, polymer type, etc.) that exhibits little to no reduction in the degree of crystallinity after milling. Combination of the above-referenced ranges are possible (e.g., at least 1.1 and less than or equal to 1000, at least 1.5 and less than or equal to 500, or at least 2 and less than or equal to 250). Other ranges are also possible.

The rate of enzymatic degradation of the crystallizable polymer or copolymer may be measured via any of a variety of appropriate methods. For example, one of more products and/or byproducts from the enzymatic degradation (e.g., depolymerization) of the crystallizable polymer or copolymer may be measured using absorbance. In some cases, the concentration of byproducts and/or products may be correlated with the measured absorbance to determine the degree of enzymatic degradation. In some cases, a titration of sodium hydroxide is used to determine the degree of enzymatic degradation during the reaction, as described elsewhere herein (See, e.g., Example 28). As an exemplary example, in embodiments in which the crystallizable polymer or copolymer comprises polyethylene terephthalate, the concentration of a specific byproduct, terephthalic acid, may be measured via absorbance and used to determine the degree of enzymatic degradation of the polymer.

In some embodiments, the method described herein may be particularly advantageous for processing a post-consumer and/or post-industrial plastic waste comprising crystallizable polymers or copolymers. For example, in some embodiments, the method comprises milling post-consumer and/or post-industrial plastic waste to reduce a percentage of crystallinity of the crystallizable polymer or copolymer contained within the post-consumer and/or post-industrial plastic waste. The milled post-consumer and/or post-industrial plastic waste, in certain embodiments, may be in the form of a plurality of polymeric particles and may have any of a variety of appropriate particle sizes and/or percentage of crystallinity described elsewhere herein (e.g. such as a crystallinity of greater than or equal to 1% and less than or equal to 12%, etc.).

Advantageously, the method described above may allow for rapid reduction in the percentage of crystallinity of post-consumer and/or post-industrial polymeric material comprising a crystallizable polymer or copolymer compared to a virgin plastic comprising the same crystallizable polymer or copolymer, under otherwise identical milling conditions. As noted earlier, the post-consumer and/or post-industrial polymeric material may be made from the virgin plastic and may additionally include some amount of additives. For example, in some embodiments, a rate of reduction of the percentage of crystallinity of the post-consumer and/or post-industrial polymeric material comprising a crystallizable polymer or copolymer is at least 1.05 times, at least 1.1 times, at least 1.2 times, 1.5 times, at least 2 times, at least 3 times, at least 5 times, at least 10 times, at least 25 times, at least 50 times, at least 75 times, or more, and/or up to 100 times, faster (or higher) than the rate of reduction of the percentage of crystallinity of a virgin plastic comprising the crystallizable polymer or copolymer, under otherwise identical milling conditions. Combination of the above-referenced ranges are possible (e.g., at least 1.1 times and less than or equal to 100 times). Other ranges are also possible.

In some embodiments, over a duration of milling, the decrease in percentage of crystallinity for a post-consumer and/or post-industrial polymeric material comprising a crystallizable polymer or copolymer is at least 1.1 times, 1.5 times, at least 2 times, at least 3 times, at least 5 times, at least 6 times, at least 9 times, at least 10 times, at least 25 times, at least 50 times, at least 75 times, or more, and/or up to 100 times, the decrease in percentage of crystallinity for a virgin plastic comprising the crystallizable polymer or copolymer. Combination of the above-referenced ranges are possible (e.g., at least 1.1 and less than or equal to 100, at least 6 and less than or equal to 100, or at least 9 and less than or equal to 100). Other ranges are also possible. The crystallizable polymer or copolymer may be milled for any appropriate durations, such as between 5 seconds and 600 seconds. In some embodiments, over a duration of milling described herein, a post-consumer and/or post-industrial polymeric material comprising the crystallizable polymer or copolymer after milling exhibits a decrease in percentage of crystallinity of at least 50%, at least 60%, at least 70%, at least 80%, and/or up to 90%, up to 95%, or up to 99% relative to its percentage of crystallinity prior to the milling. Combination of the above-referenced ranges are possible (e.g., at least 50% and up to 99%, or at least 60% and up to 90%). In some embodiments, over a duration of milling described herein, a virgin plastic comprising the crystallizable polymer or copolymer after milling exhibits a decrease in percentage of crystallinity of less than 50%, less than 40%, less than 30%, less than 20%, and/or down to 10%, down to 5%, relative to its percentage of crystallinity prior to the milling. Combination of the above-referenced ranges are possible (e.g., less than 20% and down to 5%, or less than 10% and down to 5%).

Advantageously, the method described above may allow for rapid and less energy intensive method of reduction in the percentage of crystallinity of post-consumer/post-industrial plastic particles comprising a crystallizable polymer or copolymer compared to classical methods consisting of reducing the crystallinity by fast cooling from a molten state prior to milling.

In some embodiments, the method of processing a post-consumer and/or post-industrial polymeric material further comprises combining the milled post-consumer and/or post-industrial polymeric material with an enzyme capable of facilitating degradation of the milled post-consumer and/or post-industrial polymeric material. The enzyme may include any of a variety of suitable enzymes described above.

In some embodiments, the milled post-consumer and/or post-industrial polymeric material comprising the crystallizable polymer or copolymer may be capable of being degraded by the enzyme at a relatively high rate, compared to a milled virgin plastic comprising the same crystallizable polymer or copolymer, under otherwise essentially identical conditions. As an example, milled flakes of post-consumer and/or post-industrial polyethylene terephthalate (PET) from bottles, according to some embodiments, may be degraded more readily than milled PET pellets used to make the PET bottles.

In some embodiments, a rate of enzymatic degradation per unit surface area of milled post-consumer and/or post-industrial polymeric material is at least at least 1.1 times, 1.15 times, 1.5 times, at least 2 times, at least 3 times, at least 5 times, at least 10 times, at least 25 times, at least 50 times, at least 75 times, or more, and/or up to 100 times faster (or higher) than the rate of enzymatic degradation per unit surface area of milled virgin plastic, under otherwise essentially identical conditions. Combination of the above-referenced ranges are possible (e.g., at least 1.1 times and less than or equal to 100 times). Other ranges are also possible.

Essentially identical conditions, especially as applied to conditions associated with enzymatic degradation, is a concept that will be clearly understood with precision by those of ordinary skill in the art. As examples, “essentially identical conditions” embraces conditions that can change appreciably without appreciably changing a yield of enzymatic degradation over a number of otherwise identical tests. “Essentially identical conditions” does not include conditions which, if changed appreciably, appreciably changed the yield of enzymatic degradation. Conditions such as temperature of the degradation solution and/or environment, volume of the solution, agitation of the solution, content of the solution including concentration of various species and especially the enzyme, etc. May be changed and may affect appreciably, or not affect appreciably, yield of degradation over otherwise identical tests. As noted, where a condition is changed and appreciably affects yield, all other things being essentially equal, it would not fall within the category of essentially identical conditions. Surface area of material exposed to degradation conditions, for example in particular surface area per unit volume and/or unit mass of material to undergo degradation, is not an essentially identical condition because all other things being equal higher surface area per unit volume and/or unit mass (e.g., smaller particle size) will result in a higher rate of degradation.

In some embodiments, systems for processing a crystallizable polymer or copolymer and/or PC/IPM containing the crystallizable polymer or copolymer are provided. The system, in certain embodiments, may be used to implement one or more methods described elsewhere herein. In some embodiments, the system comprises a mill. The mill may include any of a variety of mills described elsewhere herein. For example, in one set of embodiments, the mill comprises a centrifugal mill. The mill, in certain embodiments, may comprise certain components and/or have certain properties that allow for efficient processing of a crystallizable polymer or copolymer and/or PC/IPM containing the crystallizable polymer or copolymer.

In some embodiments, the mill comprises a rotor. The rotor may, in certain embodiments, comprise a plurality of rotor teeth arranged along an external circumference of the rotor. The rotor may have any of a variety of suitable numbers of rotor teeth per centimeter of the external circumference of the rotor, such as between 0.01 teeth per centimeter and 1.6 teeth per centimeter, between 0.1 teeth per centimeter and 1.3 teeth per centimeter, or between 0.2 teeth per centimeter and 0.9 teeth per centimeter.

In some embodiments, the mill comprises a ring sieve arranged concentric the external circumference (adjacent the rotor teeth) of the rotor. The sieve may comprise through-holes or apertures having any appropriate geometry, including, but not limited to, trapezoidal, circular, etc. The sieve may have any appropriate mesh size, such as between 0.05 mm and 6 mm, between 0.12 mm and 1 mm, or between 0.25 mm and 0.5 mm.

In some embodiments, the mill comprises a stationary serrated plate and a rotating serrated plate separated by a tapered gap. The size of the plates may range depending on the mill, such as between 9 inches and 16 inches in radius. The gap distance may range between 0.01 inches and 0.02 inches, or between 0.02 inches and 0.05 inches, according to some embodiments.

In some embodiments, the mill comprises a stationary serrated liner, a rotating set of hammers or knives (e.g., fixed hammers, swinging hammers, or knives), and a retaining screen. The number of hammers can range, such as from 3 to 100, in some embodiments. The size of the retaining screen holes can range between 0.03 inches and 0.18 inches, according to some embodiments.

In some embodiments, compositions are provided. The compositions, in some embodiments, may be particularly advantageous for recycling and processing of crystallizable polymers or copolymers and/or PC/IPM containing crystallizable polymers or copolymers via enzymatic degradation. In some embodiments, the crystallizable polymer or copolymer is contained within a PC/IPM, e.g., such as a post-consumer and/or post-industrial polymeric material and/or a virgin plastic.

In some embodiments, the composition comprises a plurality of polymeric particles comprising milled crystallizable polymer or copolymer and/or plastic. The plurality of polymeric particles may be formed via milling using any appropriate mills and/or operating conditions described elsewhere herein, according to some embodiments. The plurality of polymeric particles, in some embodiments, may have an average percentage of crystallinity and/or particle size that facilitate enzymatic degradation. For example, the plurality of polymeric particles may have any of a variety of appropriate average percentages of crystallinity described above, such as greater than or equal to 1% and less than or equal to 90%, greater than or equal to 1% and less than or equal to 50%, or greater than or equal to 1% and less than or equal to 40%. The plurality of polymeric particles may also have any of a variety of appropriate particle sizes described above.

The composition, in some embodiments, further comprises one or more enzymes described elsewhere herein, such as an enzyme capable of facilitating degradation (e.g., depolymerization) of the plurality of polymeric particles. The composition may further one or more liquid carrier (e.g., water or aqueous solution), a buffer, etc.

Certain aspects of the present disclosure are directed to methods of processing (e.g., milling) the crystallizable polymer or copolymer and/or a PC/IPM containing the crystallizable polymer or copolymer via a (substantially) continuous operation. In some embodiments, the crystallizable polymer or copolymer and/or a PC/IPM containing the crystallizable polymer or copolymer may be milled in a mill such that a percentage of crystallinity of the crystallizable polymer or copolymer is reduced. The milled crystallizable polymer or copolymer may, in some cases, be combined with enzyme(s) such that an enzymatic depolymerization can be carried out, according to some embodiments.

In some embodiments, the method comprises controlling one or more milling conditions during the milling such that efficient milling of the crystallizable polymer or copolymer is achieved. Non-limiting examples of milling conditions include mean residence time of the polymer within the mill, mass flow rate of polymer into the mill, a mill temperature, power draw of the mill, cooling system parameters (e.g., air flow rate through the mill, configuration of air flow), design of the mill internal components (including rotor, cutters retaining screens, serrated liners, hammers), and/or an outlet air temperature of the mill. The mill temperature, in some embodiments, may include a steady-state operating temperature within the mill and/or a temperature of the polymer within the mill during milling. Without wishing to be bound by any particular theory, by controlling one or more of the above-referenced milling parameters, one can control the degree of reduction in the polymer crystallinity (in addition to polymer sizes) in a reproducible and predictable fashion, across a wide selection of different types of mills. Furthermore, operating the mill using one or more parameters may lead to efficient reduction in crystallinity of the crystallizable polymer or copolymer and/or produce polymers that can be more readily degraded by enzymes (e.g., via enzymatic depolymerization).

In some embodiments, the method comprises milling the crystallizable polymer or copolymer to reduce a percentage of crystallinity of the crystallizable polymer or copolymer in a mill. Any of a variety of appropriate types of mills may be employed. In some embodiments, certain types of mills may be particularly advantageous compared to others in milling crystallizable polymers or copolymers (via continuous operation), e.g., such as capable of reducing the percentage crystallinity of the crystallizable polymer or copolymer at a faster rate (while allowing for high yield) and/or producing milled polymers in forms that can be more readily or efficiently degraded (e.g., depolymerized) by enzyme(s). For example, in some embodiments, mills such a rotor mill (e.g., a centrifugal mill), an attrition mill, and/or a hammer mill, may be particularly advantageous in milling crystallizable polymers or copolymers compared to other types of mills, under otherwise identical milling conditions (e.g., residence time, steady-steady temperature, etc.). However, it should be understood that the present disclosure is not so limited, and that any of a variety of appropriate mills may be employed, as long as a desirable amount of reduction in crystallinity (and/or particles sizes) can be achieved using one or more milling conditions described herein during continuous operation of the mill. For example, using the method described herein, one can adapt milling parameters to control various parameters (e.g., residence time, power and cooling system) to allow for amorphization (e.g., reduction in crystallinity) of the crystallizable polymer or copolymer via efficient milling. In some embodiments, any appropriate mills that can allow for a short residence time (in one or more ranges described below) and no overheating while producing of particles having desirably small sizes (and/or crystallinity) may be employed. Some mill designs (e.g., attrition mill and/or centrifugal mill) may be particularly advantageous for operation at higher throughput conditions, according to some embodiments.

The crystallizable polymer or copolymer may reside within the mill for any of a variety of appropriate mean residence times (i.e., average residence times). Mean residence time, according to some embodiments, is the average amount of time a given amount of polymer spend within the mill (prior to exiting the mill). In some embodiments, the crystallizable polymer or copolymer may reside within the mill for a relatively short mean residence time. In some embodiments, the crystallizable polymer or copolymer may reside within the mill for a mean residence time of greater than or equal to 0.1 seconds, greater than or equal to 0.2 seconds, greater than or equal to 0.4 seconds, greater than or equal to 0.5 seconds, greater than or equal to 0.7 seconds, greater than or equal to 1 second, greater than or equal to 2 seconds, greater than or equal to 4 seconds, greater than or equal to 6 seconds, greater than or equal to 7 seconds, greater than or equal to 7.7 seconds, greater than or equal to 8 seconds, greater than or equal to 10 seconds, greater than or equal to 30 seconds, greater than or equal to 1 minute, greater than or equal to 2 minutes, greater than or equal to 5 minutes, greater than or equal to 10 minutes, greater than or equal to 15 minutes, greater than or equal to 20 minutes, or more. In some embodiments, the crystallizable polymer or copolymer may reside within the mill for a mean residence time of less than or equal to 30 minutes, less than or equal to 20 minutes, less than or equal to 15 minutes, less than or equal to 10 minutes, less than or equal to 5 minutes, less than or equal to 2 minutes, less than or equal to 1 minute, less than or equal to 30 seconds, less than or equal to 10 seconds, less than or equal to 8 seconds, less than or equal to 7.7 seconds, less than or equal to 7 seconds, less than or equal to 5 seconds, less than or equal to 2.5 seconds, less than or equal to 1 second, less than or equal to 0.8 seconds, less than or equal to 0.75 seconds, less than or equal to 0.5 seconds, less than or equal to 0.4 seconds, or less. Combinations of the above-referenced ranges are possible (e.g., greater than or equal to 0.2 seconds and less than or equal to 30 minutes, greater than or equal to 0.2 seconds and less than or equal to 5 minutes, greater than or equal to 0.2 seconds and less than or equal to 1 minute, greater than or equal to 0.2 seconds and less than or equal to 10 seconds, or greater than or equal to 0.7 seconds and less than or equal to 7.7 seconds). Other ranges are also possible.

In some embodiments, the desirable mean residence time may be, at least in part, dictated by the power (which is associated with heat generation) and cooling system parameters associated with the mill. In some embodiments, the milled crystallizable polymers or copolymers (e.g., in the form plurality of milled particles) may have a distribution of mean residence times, e.g., with larger particles exiting the mill more quickly (e.g., having a shorter mean residence time) and smaller particles exiting the mill slower (e.g., having a longer mean residence time). Without wishing to be bound by any particular theory, it is hypothesized that milled particles having larger sizes may have a higher degree of crystallinity compared to particle having smaller sizes. The particles may have any of a variety of average sizes described elsewhere herein.

The mean residence time can be measured as follows. Firstly, the mill may be run at idle condition at a specific speed (or rotation per minute) without any polymer within the mill. A predetermined amount (i.e., a pulse mass) of crystallizable polymer or copolymer may be next introduced into the mill to cause a spike in the power draw of the mill. The time it takes for the spike in power draw to return to the value at idle condition can then be determined. The above-referenced measurement may be repeated three times. The mean residence time can be determined from an average of the times it takes for the spike in power draw to return to the value at idle condition for the three trials. The mean residence time may have any of a variety of appropriate standard deviations, such as a deviation of no more than ±0.5%, no more than ±1%, no more than ±5%, no more than ±10%, no more than ±15%, no more than ±20%, or no more than ±30% of the mean residence time.

In some embodiments, the pulse mass introduced into the mill may be greater than or equal to 0.05 grams, greater than or equal to 0.1 grams, greater than or equal to 0.2 grams, greater than or equal to 0.5 grams, greater than or equal to 1 gram, greater than or equal to 2 grams, greater than or equal to 4 grams, greater than or equal to 6 grams, greater than or equal to 8 grams, greater than or equal to 10 grams, greater than or equal to 50 grams, greater than or equal to 70 grams, greater than or equal to 100 grams, or greater than or equal to 150 grams, or more. In some embodiments, the pulse mass introduced into the mill may be less than or equal to 250 grams, less than or equal to 200 grams, less than or equal to 150 grams, less than or equal to 100 grams, less than or equal to 70 grams, less than or equal to 50 grams, less than or equal to 25 grams, less than or equal to 10 grams, less than or equal to 8 grams, less than or equal to 6 grams, less than or equal to 4 gram, less than or equal to 2 grams, less than or equal to 1 gram, less than or equal to 0.5 grams, less than or equal to 1 gram, less than or equal to 1 gram, or less. Combinations of the above-referenced ranges are also possible (e.g., greater than or equal to 0.1 grams and less than or equal to 200 grams, greater than or equal to 0.1 grams and less than or equal to 100 grams, greater than or equal to 0.1 grams and less than or equal to 70 grams, or greater than or equal to 0.1 grams and less than or equal to 6 grams). Other ranges are also possible.

The crystallizable polymer or copolymer and/or plastic containing the crystallizable polymer or copolymer may be introduced into the mill at any of a variety of appropriate apparent mass flow rates. In some embodiments, the crystallizable polymer or copolymer and/or PC/IPM containing the crystallizable polymer or copolymer may flow into the mill at a relatively high mass flow rate. For example, in some embodiments, the crystallizable polymer or copolymer and/or plastic containing the crystallizable polymer or copolymer may be introduced into the mill at an apparent mass flow rate of greater than or equal to 0.01 kg/h, greater than or equal to 0.1 kg/h, greater than or equal to 0.2 kg/h, greater than or equal to 0.4 kg/h, greater than or equal to 0.5 kg/h, greater than or equal to 0.6 kg/h, greater than or equal to 0.8 kg/h, greater than or equal to 1.0 kg/h, greater than or equal to 1.2 kg/h, greater than or equal to 1.4 kg/h, greater than or equal to 1.6 kg/h, greater than or equal to 1.7 kg/h, greater than or equal to 2.0 kg/h, greater than or equal to 5.0 kg/h, greater than or equal to 10 kg/h, greater than or equal to 50 kg/h, greater than or equal to 90 kg/h, greater than or equal to 120 kg/h, greater than or equal to 150 kg/h, or more. For example, in some embodiments, the crystallizable polymer or copolymer and/or plastic containing the crystallizable polymer or copolymer may be introduced into the mill at an apparent mass flow rate of less than or equal to 200 kg/h, less than or equal to 150 kg/h, less than or equal to 120 kg/h, less than or equal to 90 kg/h, less than or equal to 50 kg/h, less than or equal to 10 kg/h, less than or equal to 5.0 kg/h, less than or equal to 2.0 kg/h, less than or equal to 1.7 kg/h, less than or equal to 1.6 kg/h, less than or equal to 1.4 kg/h, less than or equal to 1.2 kg/h, less than or equal to 0.8 kg/h, less than or equal to 0.6 kg/h, less than or equal to 0.5 kg/h, less than or equal to 0.4 kg/h, or less. Combinations of the above-referenced ranges are possible (e.g., greater than or equal to 0.4 kg/h and less than or equal to 0.6 kg/h, or greater than or equal to 1.7 kg/h and less than or equal to 2.0 kg/h, greater than or equal to 1 kg/h and less than or equal to 200 kg/h, greater than or equal to 10 kg/h and less than or equal to 120 kg/h, or greater than or equal to 10 kg/h and less than or equal to 120 kg/h, etc.). Other ranges are also possible.

In some embodiments, the apparent mass flow rate of crystallizable polymer or copolymer and/or plastic containing the crystallizable polymer or copolymer may depend, at least in part, the particular type of mill and/or specific mill properties (e.g., dimensions). For example, in embodiments in which a centrifugal is employed, the apparent mass flow rate may have one more of the above-referenced ranges (e.g., greater than or equal to 0.4 kg/h and less than or equal to 0.6 kg/h, or greater than or equal to 1.7 kg/h and less than or equal to 2.0 kg/h, etc.). Alternatively, in embodiments in which a centrifugal is employed, the apparent mass flow rate may have one more of the above-referenced ranges (e.g., greater than or equal to 1 kg/h and less than or equal to 200 kg/h, greater than or equal to 10 kg/h and less than or equal to 120 kg/h, or greater than or equal to 10 kg/h and less than or equal to 120 kg/h, etc.). Other rangers are also possible.

Apparent mass flow rate, according to some embodiments, can be calculated by dividing the mean residence time a given amount of polymer spend within the mill by the mass of the given amount of polymer (i.e., pulse mass) initially introduced into the mill. For example, in a given mill (e.g., centrifugal mill), the apparent mass flow rate for a pulse mass of 0.2 grams of polymer and a mean residence time of 0.7 seconds is about 1.0 kg/h, according to some embodiments. For another example, in a given mill (e.g., an attrition mill), the apparent mass flow rate for a pulse mass of 20 grams and a residence time of 1.85 seconds is about 38.9 kg/h, according to some embodiments.

In some embodiments, the mass flow rate of crystallizable polymer or copolymer and/or plastic containing the crystallizable polymer or copolymer into the mill may, at least in part, affect the operating temperature within the mill and/or temperature of the polymer within mill. In one set of embodiments, by flowing the crystallizable polymer or copolymer at one or more mass flow rate described herein, a steady-state operating temperature may be achieved within the mill during continuous operation. Conversely, operating outside of the one or more mass flow rates may result in significant change in operating temperature within the mill and/or lead to operating failure of the mill (e.g., caused by film formation and/or agglomeration of polymer within the mill).

During continuous operation, the mill may be operated at any of a variety of suitable steady-state temperatures, according to some embodiments. In some embodiments, the mill may be operated at a steady-state temperature of greater than or equal to 40° C., greater than or equal to 42.5° C., greater than or equal to 44° C., greater than or equal to 45° C., greater than or equal to 47.5° C., greater than or equal to 49° C., greater than or equal to 50° C., greater than or equal to 52.5° C., or more. In some embodiments, the mill may be operated under a steady-state temperature of less than or equal to 55° C., less than or equal to 52.5° C., less than or equal to 50° C., less than or equal to 49° C., less than or equal to 47.5° C., less than or equal to 45° C., less than or equal to 44° C., less than or equal to 42.5° C., or less. Combinations of the above-referenced ranges are possible (e.g., greater than or equal to 40° C. and less than or equal to 55° C., greater than or equal to 49° C. and less than or equal to 55° C., or greater than or equal to 44° C. and less than or equal to 55° C.). Other ranges are also possible.

The “steady state” temperature of the mill will be understood by those of ordinary skill in the art based upon this disclosure and examples provided below. It is not necessarily a single precise unchanged temperature, but a temperature which generally reaches a plateau with minor fluctuation due to localized mill conditions. Additionally, it is the temperature achieved within the mill itself, and possibly, but not necessarily, reached by material being milled. The mill temperature may be determined by measuring the temperature at any suitable stationary part within the mill using a temperature sensor and/or probe over a period of time (after the initial startup of the process). For example, according to some embodiments, for a given apparent mass flow rate, the steady-state temperature within the mill may be determined by measuring the temperature of a location on a sieve within the mill or in the air in different locations within the mill. For a given apparent mass flow rate, the steady-state temperature may be determined as an average of instantaneous temperatures measured over a period of time. During continuous operation of the mill, the instantaneous temperature measured at any given point in time deviates no more than ±0.5%, no more than ±1%, no more than ±5%, no more than ±10%, or no more than ±20% from the steady-state temperature, according to some embodiments.

In some embodiments, during continuous operation, the mill may be operated such that the average temperature of the polymer within the mill is maintained at greater than (or equal to) a particular temperature or range. This temperature (of the polymer within the mill) may differ from the steady-state temperature of the mill, described above, or may be essentially the same in some situations. The polymer temperature generally will be consistent across all of the polymer and consistent within particles of the polymer itself, although small variations in temperature might exist based on mill conditions as would be understood by those orders. Wherever “polymer temperature” is used herein, it is to be understood that this is the average polymer temperature within the mill.

In some embodiments, the mill temperature (e.g., steady-state temperature and/or the average polymer temperature) can be less than or equal to 100° C., less than or equal to 90° C., less than or equal to 80° C., less than or equal to 70° C., less than or equal to 60° C., less than or equal to 50° C., less than or equal to 40° C., less than or equal to 30° C., less than or equal to 10° C., less than or equal to 5° C., or less than or equal to 2° C. above the glass transition temperature (T_{g}) of the crystallizable polymer or copolymer. In some embodiments, the mill temperature (e.g., steady-state temperature and/or the average polymer temperature) may be greater than or equal to 0° C., greater than or equal to 2° C., greater than or equal to 5° C., greater than or equal to 10° C., greater than or equal to 20° C., greater than or equal to 30° C., greater than or equal to 40° C., greater than or equal to 50° C., greater than or equal to 60° C., greater than or equal to 70° C., greater than or equal to 80° C., or greater than or equal to 90° C. above the glass transition temperature (T_{g}) of the polymer. Combinations of the above-referenced ranges are possible (e.g., greater than T_{g} and less than or equal to T_{g}+100° C., greater than T_{g} and less than or equal to T_{g}+50° C., or greater than T_{g}+50° C. and less than or equal to T_{g}+100° C.). Other ranges are also possible.

In some embodiments, the mill may be operated at a relatively low milling temperature (e.g., steady-state temperature within the mill and/or temperature of the polymer during milling) such that additional (external) cooling is not needed during the milling process. The method described herein (e.g., milling), according to some embodiments, may advantageously allow for more energy efficient pre-processing of crystallizable polymers or copolymers (at a lower operating temperature) compared to classical pre-processing method that employs high-temperature extrusion and additional cooling.

In some embodiments, it may be particularly advantageous to operate the mill at a relatively high throughput (e.g., apparent mass flow rate), a relatively low mean residence time of the polymer within the mill, and/or a relatively low milling temperature (e.g., steady-state operating temperature), as described elsewhere herein. Such a combination of milling conditions may lead to enhanced reduction in percentage of crystallinity of the crystallizable polymer or copolymer and/or produce polymers more prone for enzymatic depolymerization. Without wishing to be bound by any particular theory, it is hypothesized that (additional) decrease of recalcitrance against enzymatic degradation of milled crystallizable polymers or copolymers and PC/IPM containing crystallizable polymers or copolymers may, at least in part, be associated with a decrease in the degree of crystallinity of the crystallizable polymers or copolymers during the milling process. For example, a relatively short residence time may allow for both an increased throughput and desirable qualities in the polymer powder produced, e.g., that is, small particle sizes and more amorphous structure. The milled crystallizable polymer or copolymer may have any of a variety of particles sizes described elsewhere herein.

In some embodiments, the milled crystallizable polymer or copolymer produced by using one or more milling conditions described above may exhibit a reduction in percentage of crystallinity of the crystallizable polymer or copolymer, as described in one or more ranges elsewhere herein. In some embodiments, the milled crystallizable polymer or copolymer (e.g., in the form of a plurality of polymeric particles) produced by using one or more milling conditions described above may have a crystallinity that is at least 1%, at least 2%, at least 3%, at least 5%, at least 10%, at least 20%, at least 30%, at least 40%, at least 50%, or more, and/or up to 60%, up to 70%, up to 80%, up to 90%, up to 95%, up to 97%, or up to 99% less than the crystallinity of the crystallizable polymer or copolymers produced without using the one or more milling conditions described above. Any of the above-referenced ranges are possible (e.g., at least 1% and up to 99%, at least and up to 99%, at least 10% and up to 99%, or at least 30% and up to 99%, etc.). Other ranges are also possible. The milled crystallizable polymer or copolymer may be combined (and reacted) with one or more enzymes using methods described elsewhere herein, according to some embodiments.

In some embodiments, the milled crystallizable polymer or copolymer produced using one or more milling conditions described above (e.g., mean residence time, apparent mass flow rate, steady-state temperature) may have a relatively higher rate of enzyme degradation compared to milled crystallizable polymer or copolymer produced without using the one or more milling conditions described above. In some embodiments, a rate of enzymatic degradation per unit equivalent surface area of the milled crystallizable polymer or copolymer produced using the one or more milling conditions described above is at least 1.05 times, at least 1.1, at least 1.15, at least 1.5 times, at least 2 times, at least 3 times, at least 5 times, at least 10 times, at least 25 times, at least 50 times, at least 75 times, or more, and/or up to 100 times, up to 250 times, up to 500 times, or up to 1000 times, faster (or higher) than the rate of enzymatic degradation per unit equivalent surface area of milled crystallizable polymer or copolymer produced without using the one or more milling conditions described above. Equivalent surface area can be calculated as the surface area of a spherical particle having diameter equal to the (measured) average particle size of the corresponding crystallizable polymer or copolymer. Combination of the above-referenced ranges are possible (e.g., at least 1.05 and less than or equal to 1000, at least 1.5 and less than or equal to 500, or at least 2 and less than or equal to 250). Other ranges are also possible.

In some embodiments, the method of processing a crystallizable polymer or copolymer and/or PC/IPM containing the crystallizable polymer or copolymer, further comprises separating each of the plurality of polymeric particles by composition after milling the crystallizable polymer or copolymer and/or the PC/IPM. The separation of each of the plurality of polymeric particles by composition, in some embodiments, can advantageously improve the yield and reaction rate of enzymatic degradation. In some embodiments, any of a myriad of separation technologies can be implemented to separate the plurality of polymeric particles by composition. The separation of the milled crystallizable polymer or copolymer comprises, in some embodiments, placing the milled crystallizable polymer or copolymer into a vessel comprising water and/or an aqueous solution. In some embodiments, the aqueous solution comprises salt water and/or seawater.

When the milled crystallizable polymer or copolymer is placed into the vessel, the plurality of polymeric particles may sink and/or float at different depths within the water and/or aqueous solution. The depth at which each of the plurality of polymeric particles is located can be related to the density of each of the plurality of polymeric particles relative to that of the water and/or aqueous solution (e.g. a particle with a higher density than the aqueous solution may sink; a particle with a lower density than the aqueous solution may float). In some embodiments, the depth at which each of the plurality of polymeric particles is within the aqueous solution can be dependent on the composition of each of the plurality of polymeric particles. At an industrial scale, the plurality of polymeric particles may be located at one or more depths within the aqueous solution. In some embodiments, a portion of the plurality of polymeric particle may float on the surface of the aqueous solution. The composition of the portion of the plurality of polymeric particles on the surface of the water and/or aqueous solution can, in some embodiments, may be different than that located at a higher depth within the water and/or aqueous solution. To separate and/or isolate each of the plurality of polymeric particles by composition, the plurality of polymeric particles at the surface of the water and/or aqueous solution, and/or at a specific depth can be collected.

In some embodiments, the density of the aqueous solution can be controlled so certain compositions of the plurality of polymeric particles can be more readily collected. Compositions with a higher density than that of the aqueous solution may sink, while those with a lower density may float. This phenomenon can be leveraged, in some embodiments, to separate the plurality of polymeric particles by composition. For example, an aqueous solution with a density of 1.2 g/cm³ may allow for a portion of the plurality of polymeric particles materials such as cellulose acetate (density of approximately 1.24 g/cm³), polyvinyl chloride (density of approximately 1.3 g/cm³), polyester resin (density of approximately 1.35 g/cm³), polyethylene terephthalate (density of approximately 1.39 g/cm³), to sink to the bottom of the vessel and be readily collected. However, to collect only polyethylene terephthalate, as an example, the density of the aqueous solution can be increased to 1.37 g/cm³ (e.g. by increasing the ratio of solute to solvent) so that the plurality of polymeric particles comprising other materials (in this example, cellulose acetate, polyvinyl chloride, and polyester resin) may float on the surface and/or at an equilibrium point between the surface of the aqueous solution and the bottom of the vessel while polyethylene terephthalate sinks to the bottom of the vessel for collection. In the aforementioned example, the plurality of polymeric particles collected from the bottom of the vessel may comprise a higher amount of polyethylene terephthalate than that prior to separation. Those of ordinary skill in the art would clearly understand how to improve the separation efficiency of separating the plurality of polymeric particles by composition based on the density of each of the plurality of polymeric particles, the density of the aqueous solution, and the size of the plurality of polymeric particles. It is important to note that the aforementioned separation method advantageously limits the use of solvents that may induce plasticizing effects within the plurality of polymeric particles. Such effects may reduce the yield, reaction rate, and/or general performance of enzymatic degradation processes, in some embodiments.

The following examples are intended to illustrate certain embodiments of the present invention, but do not exemplify the full scope of the invention.

### EXAMPLES

It was discovered that by controlling grinding conditions of virgin PET pellets and post-consumer and/or post-industrial PET flakes (rPET) one can significantly improve the speed and yield of depolymerization by enzymes. It was demonstrated that one can control the physical state of milled particles by adjusting milling conditions.

### Section 1:

In this section, it was observed that enzymatic activity of polymers increased with milling.

In the following examples, PET pellets were milled using two different mills and milling was performed under different conditions and milled particles were subject to enzymatic depolymerization performed under essentially identical conditions. Milled particles were sieved such that the size of particles prepared by two milling procedures was comparable and were in the range 150-300 μm. Surprisingly, it was demonstrated that by changing conditions of milling of PET pellets, milled particles could become less recalcitrant to enzyme catalyzed depolymerization. Control of milling conditions led, both, to a faster depolymerization and to a higher yield, as measured by the increase in absorbance as the product of depolymerization terephthalic acid (TPA) was produced.

### Example 1

### Example 1.1

In this example, a centrifugal mill was used. In one milling condition (1.1), PET pellets (RAMAPET N1(S) supplied by Indorama Ventures) were milled with a centrifugal mill (Retsch ZM200) at 14000 RPM, using a sieve with a mesh size of 0.25 mm and a diameter (internal diameter) of 10 cm. Indorama PET pellets were fed into the mill at room temperature at an average feed rate of 4 g/min. Feed rate was expressed as an average feed rate. Average feed rate was obtained as the ratio between the mass of sample fed into the mill and the milling time.

The milled sample was fractionated by sieving using an analytical sieve shaker (Retsch, AS200) operating at an amplitude of ≈3 mm, for 2 cycles of 10 min (20 minutes of total shaking). Fractionation was performed using stainless steel test sieves (Retsch) with a diameter of 100 mm and mesh sizes of: 300 μm, 150 μm, 100 μm and 36 μm. The micronized PET fraction obtained between the 150 μm and 300 μm mesh sizes was used for enzymatic activity essays as follows: 25 mg of PET that was milled and sieved fraction were added in a 2 mL Eppendorf with 1 mL of potassium phosphate buffer 1 M. The Eppendorf was cooled in ice. The enzymatic activity tests were performed with the genetically modified enzyme LCC ICCG. The following is the expressed amino acid sequence of LCC ICCG by E. coli, as previously reported. The LCC-ICCG enzyme had a sequence as follows:

MSNPYQRGPNPTRSALTADGPFSVATYTVSRLSVSGFGGGVIYYPTGTSL
TFGGIAMSPGYTADASSLAWLGRRLASHGFVVLVINTNSRFDGPDSRASQ
LSAALNYLRTSSPSAVRARLDANRLAVAGHSMGGGGTLRIAEQNPSLKA
AVPLTPWHTDKTFNTSVPVLIVGAEADTVAPVSQHAIPFYONLPSTTPKV
YVELCNASHIAPNSNNAAISVYTISWMKLWVDNDTRYRQFLCNVNDPAL
CDFRTNNRHCQ

27.8 μL of stock solution (60 μM) of enzyme LCC ICCG, having a molecular weight of 30 kDa, were added in the Eppendorf. Thus the concentration of enzyme in the Eppendorf was 1.67 μM (2 mg/g of PET). The Eppendorf was incubated in a Thermomixer (Eppendorf) at 65° C. with shaking at 1200 RPM.

Reaction progress was followed by measuring the absorbance by means of a Clariostar LVis plate (BMG Labtech). Aliquots of 2 μL were taken at regular time intervals. Before measuring the absorbance, the aliquots taken during the first 2 hours of incubation time were diluted by 10 in NaOH 0.5 wt. % solution. The aliquots taken at incubation times longer than 2 hours were diluted by a factor of 100 in NaOH 0.5 wt % solution. The absorbance of diluted aliquots was measured on a Clariostar microplate. Spectra were recorded between 220 and 800 nm. The reaction yield was followed as the increase of the TPA absorbance at 242 nm (corrected by the corresponding dilution factor). Absorbance of diluted aliquots was measured by duplicate and the average absorbance was taken to follow the reaction yield.

### Example 1.2

In a second milling condition (1.2), PET pellets (50 g) were placed in a beaker and the beaker was immersed into liquid nitrogen for 1 min. Then the cooled PET pellets were transferred to a Moulinex grinder (50 g, 180 W) and milled for 1 minute. The obtained powder was transferred to the beaker and cooled for 1 minute by immersing it in liquid nitrogen. Then the cooled powder was transferred to the Moulinex grinder and milled for 1 minute. The obtained milled sample was fractionated following the sieving protocol as described before milling condition (1.1). The PET enzymatic depolymerization activity was tested with the fraction in the range 150-300 μm as described in the Example 1.1.

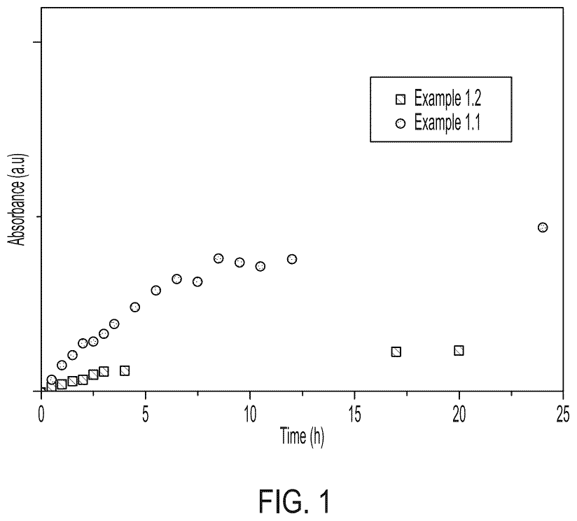

It was observed that the PET enzymatic depolymerization activity was improved by milling PET pellets with a centrifugal mill (Retsch ZM200) operating at 14000 RPM, with a sieve diameter of 10 cm and a sieve mesh of 0.25 mm. The enzymatic depolymerization activity of milled particles obtained as described in the Example 1.1-1.2 is presented in FIG. 1.

Controlling Crystallinity Degree (CD) Via Milling Conditions

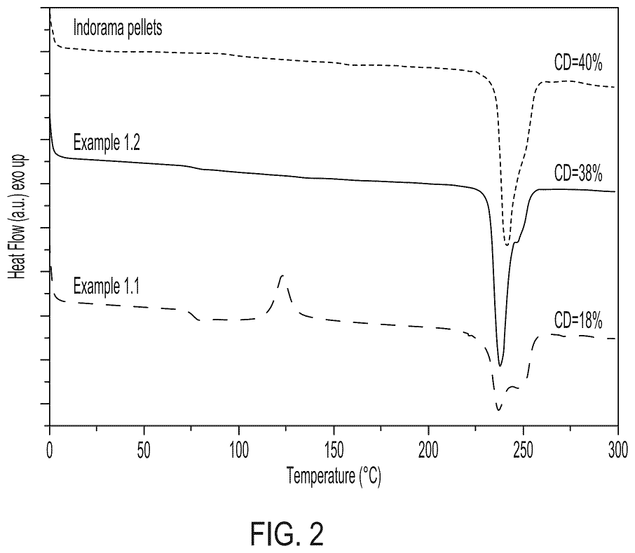

It was observed that the crystallinity degree of the polymers may be controlled by milling conditions. FIG. 2 shows the Differential Scanning Calorimetry (DSC) scans of milled PET particles, obtained by different milling conditions.

The crystallinity degree determined from DSC first heating scan (CD) is defined by Equation 1.

By milling PET under certain conditions, the crystallinity degree of particles can be reduced. Crystallinity degree characterizes the fraction of mass of crystalline phase. Without wishing to be bound by any particular theory, it is believed that the amorphous phase, compared to the crystalline phase, is more prompt for hydrolysis reactions. A decrease of crystallinity degree would imply an increase of the volume of amorphous fraction of the material and thus would lead to an increase in the rate of enzymatic depolymerization of the polymer and an increase in the depolymerization reaction yield.

The following examples illustrate how the degree of crystallinity can be decreased by controlling milling conditions.

### Example 2

In this example, PET pellets were milled using different mills. PET pellets were milled and prepared according to the protocol of the Example 1. Differential Scanning Calorimetry (DSC) first heating scan of PET samples was obtained using a calorimeter (TA, discovery Q200). 10 mg of PET were introduced in a capsule (TA, Tzero Pan T 220228 and Tzero Hermetic Lid T 220315). The first heating scan was collected following the sequence: 1) Equilibrate temperature from room temperature to 0° C.; 2) Isothermal step (0° C.) for 1 min; 3) Heating step from 0° C. to 300° C. at a heating rate of 10° C./min. From the normalized heat flow curve vs temperature, the degree of crystallinity (CD) was calculated as defined in ec.1 using the TRIOS software version v3.1.5.3696. The integration of the cold crystallization exothermic peak at around (115-135) ° C. and the melting endothermic peak at around 250° C. was performed from visually determined respective starting points to end points using a straight baseline between them to get ΔHₘₑₗₜ and ΔH_{cold crystallization}.

DSC characterization shows that the CD of the milled sample described in the Example 1.1 obtained by centrifugal milling is of 18% and the CD of milled sample described in the Example 1.2 obtained by Moulinex grinder is of 38%.

### Example 3

In this example, cryo-milling of Indorama pellets was performed. The crystallinity degree of Indorama pellets (starting material) is 40%. PET pellets (50 g) were placed in a beaker and the beaker was immersed into a container filled with liquid nitrogen for 2 min to cool the PET pellets. Pre-cooled PET pellets were milled with a centrifugal mill (Retsch ZM200) at 14000 RPM, using a sieve with a mesh size of 0.5 mm and a diameter of 10 cm. The pre-cooled pellets were fed into the mill at an average feed rate of 5 g/min. CD of particles did not decrease when samples were cooled prior to milling and remained at 40%. Milling pre-cooled PET pellets (cooled with liquid nitrogen) did not produce any change in the CD of milled PET.

In addition, even by milling PET from room temperature, it was not always possible to obtain PET particles. The following comparative example 4 illustrates that when feed rates were too high in the milling process, instead of forming PET particles, a PET film is formed at the inner surface of the sieve. The film blocked the holes of the sieve and the milling process was terminated.

### Comparative Example 4

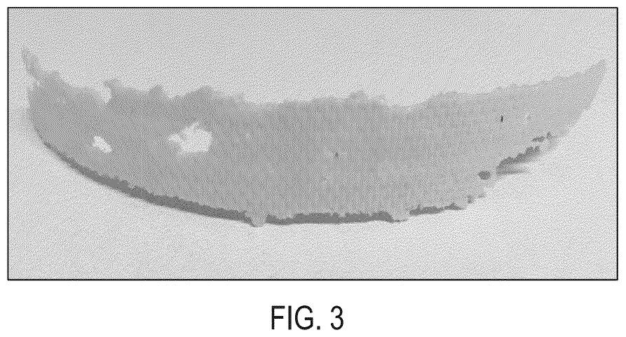

Indorama pellets were milled under the same conditions as in the Example 1.1 with the only exception that the average feed rate was increased from 4 g/min to 18 g/min. After 28 seconds of milling, the process was stopped due to the formation of a film, which was evidenced by a characteristic sound as the teeth of the rotor hit the film. The milling was immediately stopped and the formation of a film (FIG. 3) was confirmed when opening the lid of the mill. The CD of the film thus obtained was 11% and the CD of the milled PET was 19%.

### Comparative Example 5

Indorama pellets were milled under the same conditions as in the Example 1.1 with the only exception that the average feed rate was increased from 4 g/min to 9 g/min. After 160 seconds of milling, the process was stopped due to the formation of a film, as shown in FIG. 3., which was evidenced by a characteristic sound as the teeth of the rotor hit the film. The milling was immediately stopped and the formation of a film was confirmed when opening the lid of the mill. The CD of the film thus obtained was 7% and the CD of the milled PET was 19%.

### Controlling CD of Milled Particles Via Temperature Control

It was discovered that temperature control inside centrifugal mill allowed for control CD of milled particles. To avoid the formation of such films it is essential to control the temperature of the process. Temperature control was achieved by adjusting the feed rate. A very high feed rate conducted to film formation (blockage). Temperature was monitored at the surface of the sieve by sticking a type K thermocouple to the external wall of the sieve.

Temperature control could also be achieved by evacuating the heat formed during milling. In addition, the following parameters were found to be also relevant to control the temperature of the milling process.

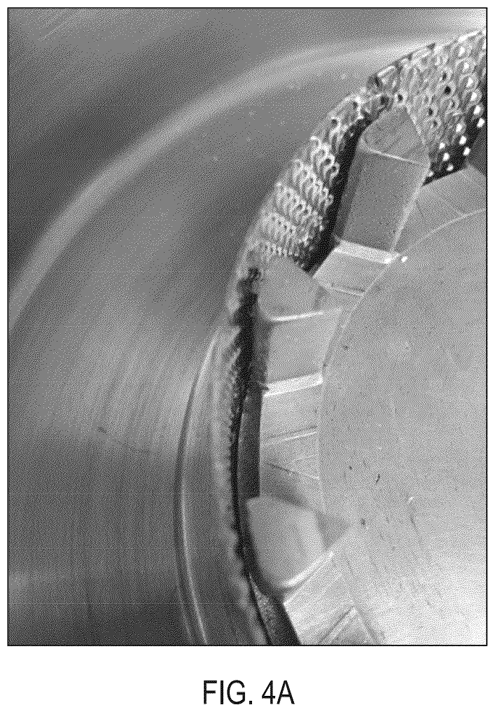

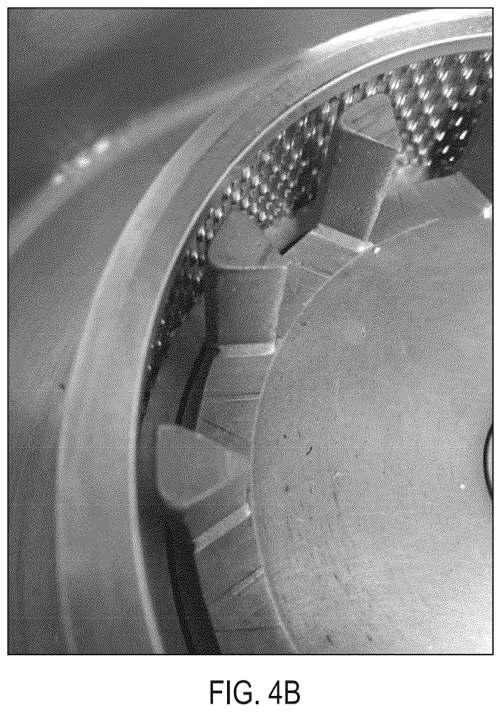

Parameter (i) Teeth-to-sieve gap (see FIG. 4A and FIG. 4B): The distance between the teeth of the rotor and the sieve is an important parameter that can be adjusted to better control the temperature of the process at a certain feed rate. In the examples given in Table 1, there are two teeth-to-sieve gaps: Small gap (S) using a sieve diameter of 10 cm or Large gap (L) using a sieve diameter of 11.3 cm. Example 5 illustrates milling using different teeth-to-sieve gaps.

### Example 5

### Example 5.1

In one milling condition (5.1), PET pellets (50 g) were milled with a centrifugal mill (Retsch ZM200) at 14000 RPM, using a sieve with a mesh size of 0.5 mm and a diameter of 10 cm (small teeth-to-sieve gap). Indorama PET pellets were fed into the mill at room temperature at an average feed rate of 5 g/min during 10 min. The temperature of the sieve was monitored during the milling time. The maximum temperature was 63° C. and the crystallinity degree of the micronized PET was 36%.

### Example 5.2

In a second milling condition (5.2), PET pellets (50 g) were milled with a centrifugal mill (Retsch ZM200) at 14000 RPM, using a sieve with a mesh size of 0.5 mm and a diameter of 11.3 cm (large teeth-to-sieve gap). Indorama PET pellets were fed into the mill at room temperature at an average feed rate of 3.1 g/min during 16 min. The temperature of the sieve was monitored during the milling time. The maximum temperature was 155° C. and the crystallinity degree of the micronized PET was 24%.

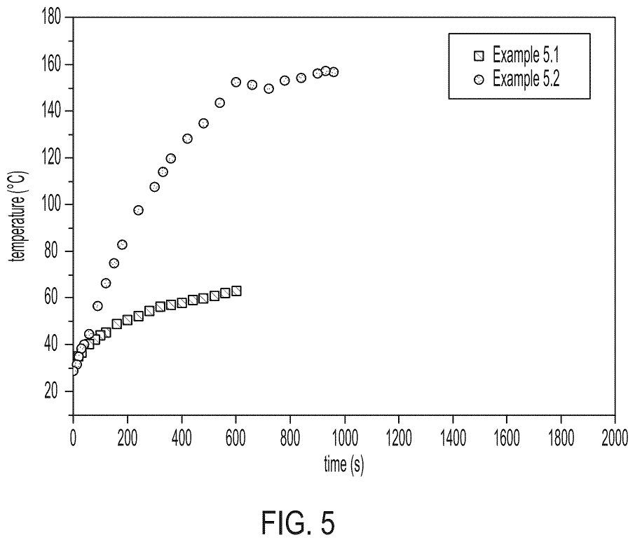

FIG. 5 shows the evolution of temperature of the sieve during the milling conditions described in the Example 5.

Parameter (ii) Mesh size. The size of the holes of the sieves was another important parameter. Using a higher mesh size can allow for an increase the feed rate. In the examples given in Table 1, there are three mesh sizes expressed in mm: 1.0; 0.5 and 0.25. Not all the mesh sizes led to a decrease of CD after milling (example of 1.0 mm). Example 6 illustrates milling using various mesh sizes.

### Example 6

### Example 6.1

In one milling condition (6.1), PET pellets were milled at 14000 RPM using a small teeth-to-sieve gap and a sieve mesh size of 0.25 mm. The pellets were fed into the mill at room temperature at an average feed rate of 4 g/min. The temperature of the sieve was monitored during the milling time. The maximum temperature was 100° C. and the CD of the micronized PET was 18%.

### Example 6.2

In a second milling condition (6.2), PET pellets were milled at 14000 RPM using a small teeth-to-sieve gap and a sieve mesh size of 0.5 mm. The pellets were fed into the mill at room temperature at an average feed rate of 5 g/min. The temperature of the sieve was monitored during the milling time. The maximum temperature was 63° C. and the CD of the micronized PET was 36%.

### Example 6.3

In a third milling condition (6.3), PET pellets were milled at 14000 RPM using a small teeth-to-sieve gap and a sieve mesh size of 1.0 mm. The pellets were fed into the mill at room temperature at an average feed rate of 5 g/min. The temperature of the sieve was monitored during the milling time. The maximum temperature was 47° C. and the CD of the micronized PET was 40%.

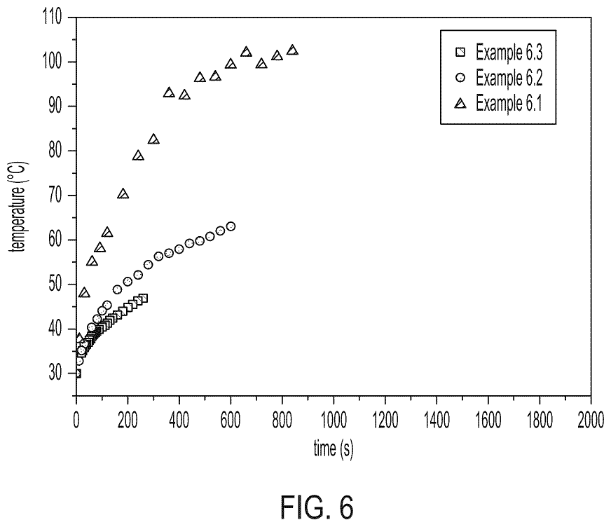

FIG. 6 shows the evolution of temperature of the sieve during the milling conditions described in the Example 6.

Parameter (iii) Temperature of feed material is a relevant parameter of milling conditions. The following example illustrates how the choice of temperature of feed material impacts in the temperature of milling and thus in CD.

### Example 7

### Example 7.1

In one milling condition (7.1), PET pellets were milled at 14000 RPM using a small teeth-to-sieve gap and a sieve mesh size of 0.5 mm. The pellets were fed into the mill at room temperature at an average feed rate of 5 g/min. The temperature of the sieve was monitored during the milling time. The maximum temperature was 63° C. and the CD of the micronized PET was 36%.

### Example 7.2

In a second milling condition (7.2), pre-cooled PET pellets were milled at 14000 RPM using a small teeth-to-sieve gap and a sieve mesh size of 0.5 mm, following the same procedure as in the Example 3. The temperature of the sieve was monitored during the milling time. The maximum temperature was 54° C. and the CD of the micronized PET was 40%.

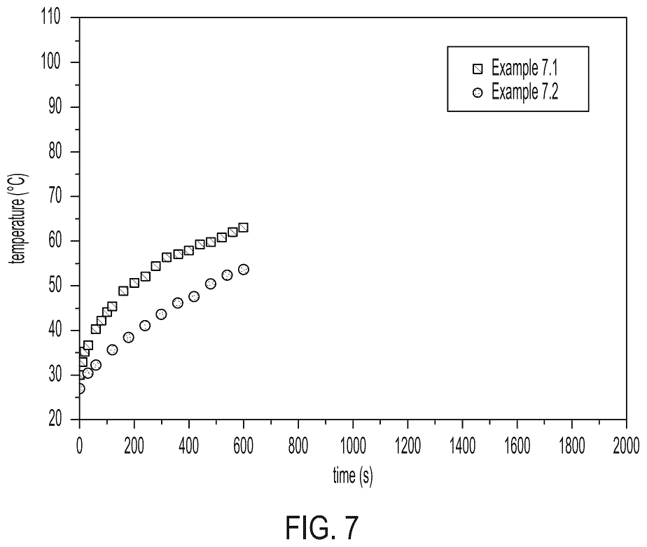

FIG. 7 shows the evolution of temperature of the sieve during the milling conditions described in the Example 7.

TABLE 1
Milling conditions for PET

| | | Temper- | Average | | | Crystal- |
|---|---|---|---|---|---|---|
| | | ature | feed | T | T | linity |
| Exam- | Milling | gradient | rate | “plateau” | initial | degree |
| ple | condition | (° C./min) | (g/min) | (° C.) | (° C.) | (%) |
| 6.3 | 1.0_S_RT | 4 | 5 | 47 (max.) | 30 | 40 |
| 5.1 | 0.5_S_RT | 3.3 | 5 | 63 (max.) | 30 | 36 |
| 6.1 | 0.25_S_RT | 10 | 4 | 100 | 34 | 18 |
| 5.2 | 0.5_L_RT | 12.4 | 3.1 | 155 | 28 | 24 |
| 7.2 | 0.5_S_N2 | 3 | 5 | 54 (max.) | 27 | 40 |

The following operating parameters and their ranges are relevant to control the performance of milling: such (i) a temperature “plateau” in the range of 0-250° C., with a preferred range 15-250° C.; (ii) a temperature gradient in the range of 0.1-50° C./min, and/or (i) a feed rate in the range of 0.2 g/min-50 kg/min.
Parameter (iv) RPM: For a centrifugal mill, the RPM can be adjusted to modify the feed rate at a given mill dimension. This parameter should be of use to improve the milling throughput and may be important for scaling-up purposes. In the previous examples an RPM of 14000 was employed. For a higher dimension mill (higher rotor and sieve diameter) the RPM may be decreased accordingly relative to a laboratory scale centrifugal mill.

### Section 2.

It was discovered that by controlling grinding conditions of post-consumer PET flakes, it is also possible to significantly increase the rate and yield of depolymerization by enzymes (measured as the increase of the TPA absorbance). Surprisingly, it was observed that by selecting the proper milling conditions, the effect is even more pronounced than for the case of virgin PET pellets, as described in Example 1.

In the following example, a starting material rPET that was non-homogeneous was milled:

The post-consumer PET flakes (rPET) purchased from PolyQuest is a non-homogenous material having different sizes (thickness), mixed colors as it refers to ground washed bottle flakes from beverage, food, and non-food packaging. Since the different parts of the PET plastic bottles (neck, shoulder, body, etc.) were not homogeneous, it was expected that the crystallinity degree of rPET flakes would vary from flake to flake. For this reason and to have a better characterization of the average CD of the staring material, DSC experiments were performed on different flakes and on cryo-milled rPET.

The following example illustrates the variability of CD of rPET flakes:

A thick flake (thickness of 1.5 mm) from rPET was cut with a scissor and characterized by DSC first heating scan. The CD of the thick flake sample was 11%.

A thin flake (thickness of 0.2-0.4 mm) was randomly picked from the rPET sample and cut with a scissor. The cuts of rPET thin flake were characterized by DSC. The CD of the thin flake was 27%.

rPET (50 g) were placed in a beaker and pre-cooled in liquid nitrogen for 1 min. 15 Then the pre-cooled rPET flakes were transferred to a Moulinex grinder (50 g, 180 W) and milled for 1 min. The cooling step and milling step were repeated 4 times to give a total milling time of 4 min. The obtained fragments were characterized by DSC (by triplicate) to estimate an average crystallinity degree of rPET sample. The CD was of the fraction was (24±2)%.

Enzymatic Activity Increased by Milling of rPET Flakes:

In the following example rPET flakes were milled using two different mills and milling was performed under different conditions and milled particles were subject to enzymatic depolymerization performed under essentially identical conditions. Milled particles were sieved so that the size of particles prepared by two milling procedures was comparable and were in the range 150-300 m. Surprisingly, it was discovered that by changing conditions of milling of rPET pellets, milled particles can become less recalcitrant to enzyme catalyzed depolymerization. Control of milling conditions leads, both, to a faster depolymerization and to a higher the yield (measured as the increase of the TPA absorbance).

By controlling milling conditions of post-consumer PET flakes, the yield of depolymerization by enzymes (measured as the increase of the TPA absorbance) was significantly increased.

### Example 8

In this example, rPET milled using centrifugal mill was compared to rPET milled using Moulinex mill

### Example 8.1

In one milling condition (8.1), rPET flakes (25 g) were milled with a centrifugal mill (Retsch ZM200) at 14000 RPM, using a sieve with a mesh size of 0.25 mm and a diameter of 10 cm (small teeth-to-sieve gap). rPET flakes were fed into the mill at room temperature at an average feed rate of 1.6 g/min. The milled sample was fractionated following the sieving protocol as described before in the Example 1. The PET enzymatic depolymerization activity was tested with the fraction in the range 150-300 m following the same procedure as described in the Example 1.

### Example 8.2

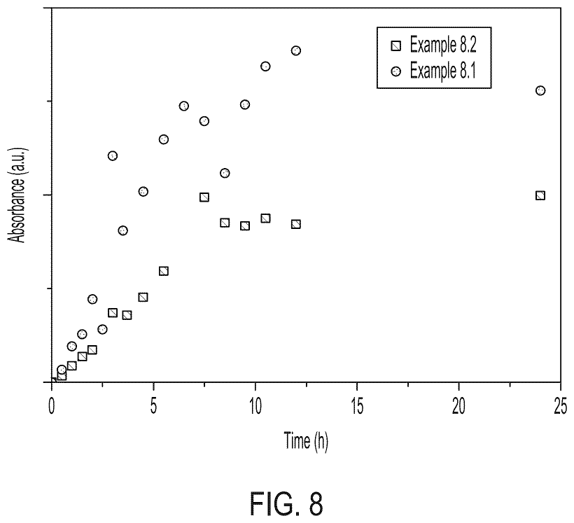

In a second milling condition (8.2), rPET flakes (50 g) were placed in a beaker and pre-cooled in liquid nitrogen for 1 min. Then the pre-cooled rPET flakes were transferred to a Moulinex grinder (50 g, 180 W) and milled for 1 min. The cooling step and milling step were repeated 4 times to give a total milling time of 4 min. The obtained milled sample was fractionated following the sieving protocol as described in the Example 1. The PET enzymatic depolymerization activity was tested with the fraction in the range 150-300 m as described in the Example 1. The enzymatic depolymerization activity of milled particles of the Example 8.1 and 8.2 is presented in FIG. 8.

### Example 9

In this example, Evian milled using Moulinex was compared to Evian milled using a centrifugal mill

### Example 9.1

In one milling condition (9.1), the main body of an Evian mineral water bottle was cut with a scissor into flakes with sizes in the range (0.5-0.8) mm. Evian flakes (5 g) were milled with a centrifugal mill (Retsch ZM200) at 14000 RPM, using a sieve with a mesh size of 0.5 mm and a diameter of 10 cm (small teeth-to-sieve gap). Evian flakes were fed into the mill at room temperature at an average feed rate of 3.3 g/min. The milled sample was fractionated following the sieving protocol as described before in the Example 1. The PET enzymatic depolymerization activity was tested with the fraction in the range 150-300 m following the same procedure as described in the Example 1.

### Example 9.2

In a second milling condition (9.2), Evian bottle flakes were prepared as described in the Example 9.1. Evian Flakes (10 g) were placed in a beaker and pre-cooled in liquid 10 nitrogen for 1 min. Then the pre-cooled rPET flakes were transferred to a Moulinex grinder (50 g, 180 W) and milled for 1 min. The cooling step and milling step were repeated 4 times to give a total milling time of 4 min. The obtained milled sample was fractionated following the sieving protocol as described in the Example 1. The PET enzymatic depolymerization activity was tested with the fraction in the range 150-300 m as described in the Example 1.

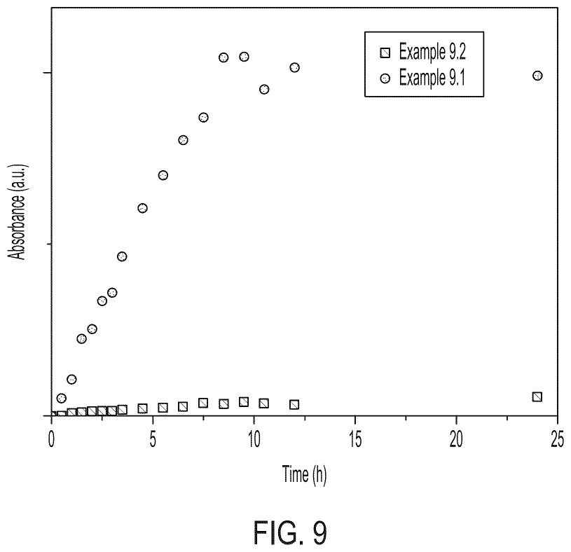

The PET enzymatic depolymerization activity was improved by milling Evian flakes with a centrifugal mill (Retsch ZM200) operating at 14000 RPM, with a sieve diameter of 10 cm and a sieve mesh of 0.25 mm. The enzymatic depolymerization activity of milled particles of the example 9.1 and 9.2 is presented in FIG. 9.

### Example 10

In this example, rPET milled using a centrifugal mill was compared to rPET milled using a Moulinex mill.

### Example 10.1

In one milling condition (10.1), rPET flakes (50 g) were milled with a centrifugal mill (Retsch ZM200) at 14000 RPM, using a sieve with a mesh size of 0.5 mm and a diameter of 10 cm (small teeth-to-sieve gap). rPET flakes were fed into the mill at room temperature at an average feed rate of 4.2 g/min. The milled sample was fractionated following the sieving protocol as described before in the Example 1. The PET enzymatic depolymerization activity was tested with the fraction in the range 150-300 m following the same procedure as described in the Example 1.

### Example 10.2

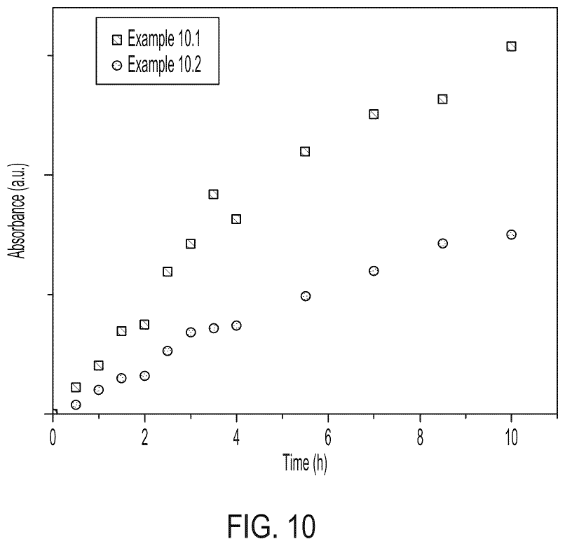

In a second milling condition (10.2), rPET flakes (50 g) were placed in a beaker and pre-cooled in liquid nitrogen for 1 min. Then the pre-colled rPET flakes were transferred to a Moulinex grinder (50 g, 180 W) and milled for 1 min. The cooling step and milling step were repeated 4 times to give a total milling time of 4 min. The obtained milled sample was fractionated following the sieving protocol as described in the Example 1. The PET enzymatic depolymerization activity was tested with the fraction in the range 150-300 m as described in the Example 1. The enzymatic depolymerization activity of milled particles of the example 10.1 and 10.2 is presented in FIG. 10.

### Control of Crystallinity Degree by Milling Conditions

The following examples illustrate how the degree of crystallinity of rPET flakes can be drastically decreased by controlling the milling conditions.

### Example 11

rPET flakes (10 g) were milled with a centrifugal mill (Retsch ZM200) at 14000 RPM, using a sieve with a mesh size of 1.0 mm and a diameter of 10 cm (small teeth-to-sieve gap). rPET flakes were fed into the mill at room temperature at an average feed rate of 2 g/min. The temperature of the sieve was monitored during the milling time. The maximum temperature was 60° C. and the CD of the micronized rPET was 11%.

### Example 12

rPET flakes (50 g) were milled with a centrifugal mill (Retsch ZM200) at 14000 RPM, using a sieve with a mesh size of 0.5 mm and a diameter of 10 cm (small teeth-to-sieve gap). rPET flakes were fed into the mill at room temperature at an average feed rate 25 of 4.2 g/min. The CD of the micronized rPET was 2%.

### Example 13

rPET flakes (25 g) were milled with a centrifugal mill (Retsch ZM200) at 14000 RPM, using a sieve with a mesh size of 0.25 mm and a diameter of 10 cm (small teeth-to-sieve gap). rPET flakes were fed into the mill at room temperature at an average feed rate of 1.6 g/min. The temperature of the sieve was monitored during the milling time. The maximum temperature was 97° C. and the CD of the micronized rPET was 6%.

It was observed that the feed rate can be used to control the effect of milling on the crystallinity degree.

The following comparative example illustrates that importance of controlling the feed rate.

### Comparative Example 14

rPET flakes were milled with the same milling conditions as in the Example 12 with the only exception that the average feed rate was increase from 4.2 g/min to 7.5 g/min. The temperature of the sieve was monitored during the milling time. After 130 seconds of milling, an abrupt increase of the temperature was evidenced, keeping the same feed rate. The milling was stopped after 170 seconds. Formation of a film in the internal wall of the sieve was evidenced when opening the lid of the mill. The CD of the film thus 15 obtained was 27% and the CD of the milled rPET was 9%.

The following example illustrates that the gap between the teeth and the sieve is an important parameter in controlling the temperature of the milling and thus the degree of crystallinity of rPET by milling. In one milling condition of the example, the choice of a small gap allows for the production of micronized rPET with a very low CD, whereas in another milling condition the choice of a large gap, even at a lower feed rate, did not produce a significant change in the CD respect to the CD of the starting rPET flakes.

### Example 15

### Example 15.1

In one milling condition (15.1), rPET flakes (50 g) were milled with a centrifugal mill (Retsch ZM200) at 14000 RPM, using a sieve with a mesh size of 0.5 mm and a diameter of 10 cm (small teeth-to-sieve gap). rPET flakes were fed into the mill at room temperature at an average feed rate of 4.2 g/min. The temperature of the sieve was monitored during the milling time. The maximum temperature was 90° C. and the CD of 30 the micronized rPET was 2%.

### Example 15.2

In a second milling condition (15.2), rPET flakes (50 g) were milled with a centrifugal mill (Retsch ZM200) at 14000 RPM, using a sieve with a mesh size of 0.5 mm and a diameter of 11.3 cm (large teeth-to-sieve gap). rPET flakes were fed into the mill at room temperature at an average feed rate of 1.5 g/min. The temperature of the sieve was monitored during the milling time. The maximum temperature was 163° C. and the CD of the micronized rPET was (22±1)%.

The same effect of milling on the control of the crystallinity degree of rPET flakes was also observed for PET flakes from bottles, as illustrated in the following example.

### Example 16

In this example, Evian flakes were milled to reduce their crystallinity.

The main body of an Evian mineral water bottle was cut with a scissor into flakes with sizes in the range (0.5-0.8) mm. The CD of the Evian flakes thus obtained was 30%. Evian flakes (5 g) were milled with a centrifugal mill (Retsch ZM200) at 14000 RPM, using a sieve with a mesh size of 0.5 mm and a diameter of 10 cm (small teeth-to-sieve gap). Evian flakes were fed into the mill at room temperature at an average feed rate of 3.3 g/min. The temperature of the sieve was monitored during the milling time. The maximum temperature was 50° C. and the CD of the micronized Evian flakes was 5%.

In Table 2, examples of different milling conditions of rPET, expressed as “mesh size_gap_temperature of feed material” was shown.

TABLE 2
Milling conditions for rPET

| | | Temperature | Average | T | T | |
|---|---|---|---|---|---|---|
| | Milling | gradient | feed rate | “plateau” | initial | Crystallinity |
| Example | condition | (° C./min) | (g/min) | (° C.) | (° C.) | degree (%) |
| 11 | 1.0_S_RT | 6 | 2 | 60 (max) | 31 | 11 |
| 12 | 0.5_S_RT | 5.5 | 4.2 | 90 | 32 | 2 |
| 13 | 0.25_S_RT | 3.6 | 1.6 | 97 | 38 | 6 |
| 15.2 | 0.5_L_RT | 18.6 | 1.5 | 163 | 25 | 22 |

The milling process was operated by controlling feed rate (controlling parameter) and monitoring temperature (monitoring parameter).

To increase the throughput of the milling process of rPET flakes and productivity, the feed rate could be increased by reducing the size of the flakes. The following example illustrates how the size reduction of rPET flakes can be used to increase feed rate, while keeping the other milling conditions as well as the maximum temperature.

### Example 17

### Example 17.1

In one milling condition (17.1), rPET flakes (10 g) were milled with a centrifugal mill (Retsch ZM200) at 14000 RPM, using a sieve with a mesh size of 0.5 mm and a diameter of 10 cm (small teeth-to-sieve gap). rPET flakes were fed into the mill at room temperature at an average feed rate of 1.8 g/min. The temperature of the sieve was monitored during the milling time. The maximum temperature was 58° C. and the CD of the micronized rPET was 9%.

### Example 17.2

In a second milling condition (17.2), rPET flakes (50 g) were placed in a beaker and pre-cooled in liquid nitrogen for 1 min. Then the pre-colled rPET flakes were transferred to a Moulinex grinder (50 g, 180 W) and milled for 1 min. The cooling step and milling step were repeated 4 times to give a total milling time of 4 min. The pre-milled rPET flakes had a size of around 0.2-0.5 mm. Pre-milled rPET flakes (10 g) were milled with a centrifugal mill (Retsch ZM200) at 14000 RPM, using a sieve with a mesh size of 0.5 mm and a diameter of 10 cm (small teeth-to-sieve gap). Pre-milled rPET were fed into the mill at room temperature at an average feed rate of 3.7 g/min. The temperature of the sieve was monitored during the milling time. The maximum temperature was 58° C. and the CD of the micronized rPET was 9%.

### Section 3.

It was discovered that by controlling the milling conditions of PET, it is possible to improve the rate and yield of enzymatic depolymerization by enzymes (measured as the increase of the TPA absorbance) without the use of any pretreatment to reduce the crystallinity degree of crystallizable polymers or copolymers prior to the milling step.

The following example illustrates that the enzymatic activity of milled PET samples obtained by centrifugal milling was comparable or even better than the one obtained by pretreatment consisting of extrusion and cooling followed by micronization in standard mills and milling conditions. Milled particles were sieved so that the size of particles prepared by two milling procedures was comparable and were in the range 150-300 mm.

### Example 18

### Example 18.1

PET by milling. In one milling condition (18.1), rPET flakes were prepared and milled as described in the Example 13. The PET enzymatic depolymerization activity was tested with the fraction in the range 150-300 μm as described in the Example 13. The CD of the milled sample was 6%.

### Example 18.2

PET by extrusion, fast cooling and milling. In a second condition (18.2), rPET flakes (14 g) were fed into a conical twin screw extruder (DSM, Xplore, 15 cm³ capacity) equipped with co-rotating conical screw profile and recirculation channel to control the residence time. The extrusion was performed under circulation of nitrogen, with a barrel temperature profile as follows. Three temperature controls at different positions of the barrel were set as follows: Top position (270° C.), middle position (270° C.) and exit position (280° C.). The speed of rotation of the screws was 60 RPM. After rPET feed (45 seconds) and a residence time of 2 minutes, the material was extruded directly into an ice water bath (5° C.) to be quenched in said bath. Transparent rPET extrudate having a diameter of (1-2) mm was characterized by DSC. The CD of rPET extrudate was 11%. rPET extrudate (10 g) was cut with a scissor to a length size of 1-3 cm and the resulting cuts were placed in a beaker and the beaker was immersed into liquid nitrogen for 1 min. Then the cooled rPET sample was transferred to a Moulinex grinder (50 g, 180 W) and milled for 1 minute. The obtained milled sample was transferred to the beaker and cooled for 1 minute by immersing it in liquid nitrogen. Then the cooled sample was transferred to the Moulinex grinder and milled for 1 min. The obtained milled sample was fractionated following the sieving protocol as described in the Example 1. The PET enzymatic depolymerization activity was tested with the fraction in the range 150-300 μm as described in the Example 1.

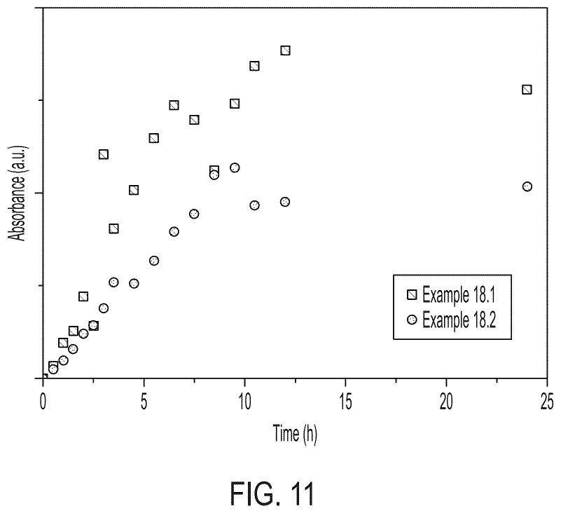

The enzymatic depolymerization activity of milled particles of the Example 18.1 and 18.2 is presented in FIG. 11.

### Section 4.

The milling process described here can be operated as a continuous process.

Here the temperature of the sieve can be defined as the monitoring parameter of a continuous milling process. By milling at a constant controlled feed rate (controlling parameter), the temperature of the sieve was observed to increase with a certain temperature gradient. After certain milling time at a constant feed rate, the steady state milling conditions were reached and the temperature of the sieve remains constant. The following examples illustrate that the milling process described here can be adapted into a continuous process.

In the following example, it was discovered that the temperature gradient can be adjusted by the feed rate.

### Example 19

rPET flakes were prepared and milled as described in the Example 15.1. The temperature of the sieve was monitored during the milling time. rPET flakes were fed at an average feed rate of 4.2 g/min. The temperature gradient was 5.5° C./min. The maximum sieve temperature was 90° C. and the CD of the micronized rPET was 2%.

### Comparative Example 20

rPET flakes were prepared and milled under essentially identical conditions as described in the Example 19 with the only exception that the feed rate was increased to 7.5 g/min. The temperature gradient was 25° C./min.

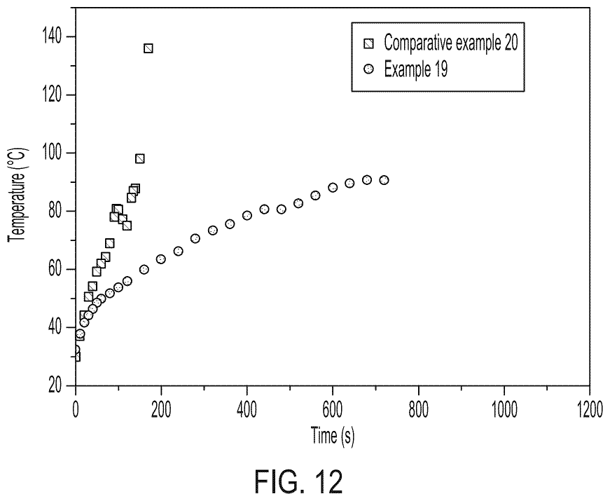

The temperature gradient can be adjusted by controlling the feed rate as presented in FIG. 12 for the conditions described in the Example 19 and the Comparative example 20.

In the following example, it was discovered that the temperature of milling can reach a plateau.

### Example 21

rPET flakes were prepared and milled as described in the Example 13. The temperature of the sieve was monitored during the milling time. The temperature gradient was 3.6° C./min. After 18 min. of continuous milling at a constant average feed rate of 1.6 g/min the temperature of the sieve reached a plateau of 97° C.

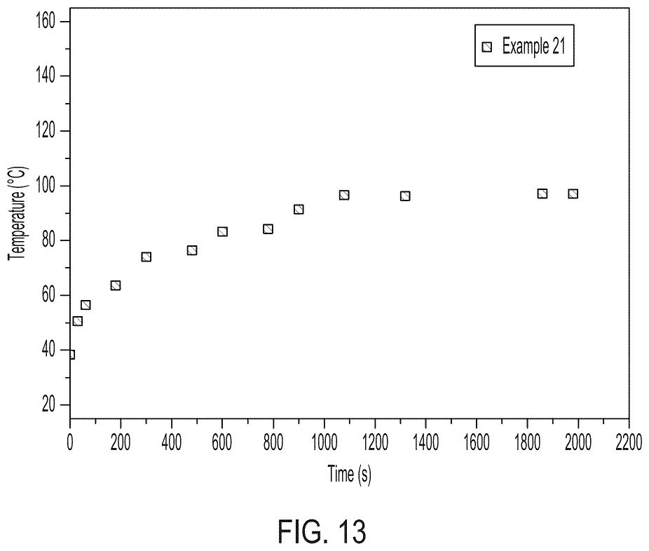

After continuous milling at a constant feed rate, the temperature of the sieve reaches a plateau as presented in FIG. 13 for the conditions described in the Example 21.

In the following example, it was discovered that the crystalline degree (CD) can be controlled by the time of milling.

### Comparative Example 22

rPET flakes were prepared and milled under essentially identical conditions as described in the Example 19 with the only exception that milling time was 5 sec. instead of 10 min, as for the Example 19. The sieve temperature vs time was identical as for the Example 19. The maximum sieve temperature was 34° C. and the CD of the milled sample was 9%.

### Comparative Example 23

rPET flakes were prepared and milled under essentially identical conditions as described in the Example 19 with the only exception that milling time was 30 sec. instead of 10 min, as for the Example 19. The sieve temperature vs time was identical as for the Example 19. The maximum sieve temperature was 47° C. and the CD of the milled sample was 10%.

### Comparative Example 24

rPET flakes were prepared and milled under essentially identical conditions as described in the Example 19 with the only exception that milling time was 1 min. instead of 10 min, as for the Example 19. The sieve temperature vs time was identical as for the Example 19. The maximum sieve temperature was 50° C. and the CD of the milled sample was 7%.

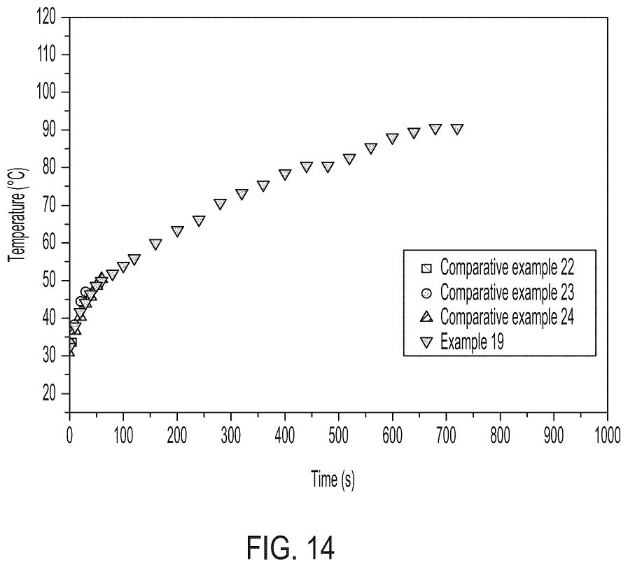

FIG. 14 shows that the sieve temperature versus time curve can be consistently reproduced by milling PET under the same milling conditions. Before reaching the plateau condition, an increase of milling time (and thus milling temperature) leads to a decrease of the CD of milled PET. Under the milling conditions described in the Example 19 a CD as low as 7% is obtained after milling for 1 min.

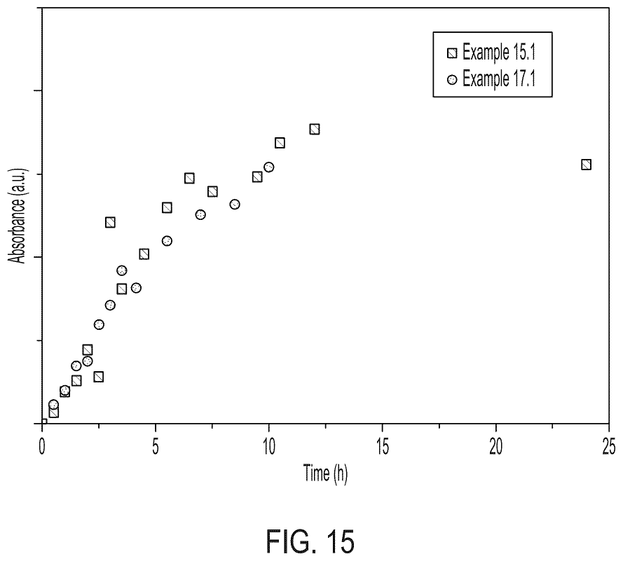

FIG. 15 shows the enzymatic depolymerization activity of milled particles described in the Example 15.1 and 17.1, having a CD of 6% and 2%, respectively. It was observed that for both conditions the rate and yield of enzymatic depolymerization by enzymes were comparable. Therefore, the milling process described here can be adapted into a continuous process in which the output material becomes less recalcitrant for enzymatic depolymerization from certain (short) milling times.

In a continuous milling process, the output material should be constantly withdrawn at a rate compatible with the feed rate. The latter could be achieved with a suitable mill design.

### Section 5:

### Process Ranging of PET Milling

The goal of these experiments was to identify key process parameters for rotor milling PET pellets in preparation for enzyme-catalyzed depolymerization. All work was performed on a Retsch ZM200 rotor mill with PET pellets (RAMAPET N1(S) supplied by Indorama Ventures).

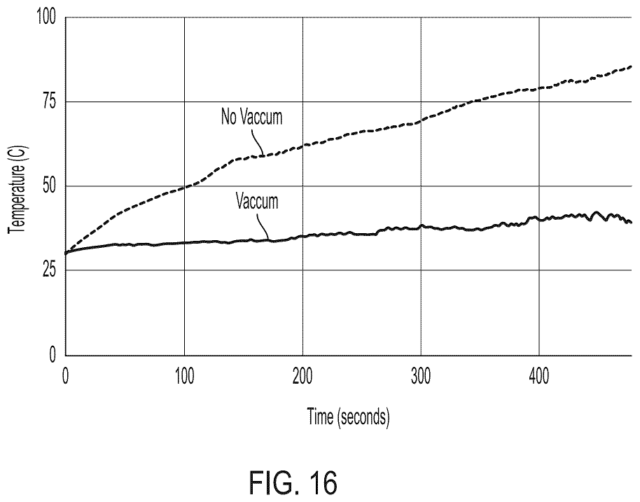

The first goal was to determine the effect of evacuation air (vacuum) on process conditions. Temperature was measured within the receiving bowl of Retsch mill using a standard thermocouple. The temperature without vacuum climbs steadily as seen in FIG. 16. When the temperature inside the rotor mill reaches the melting temperature of PET, a film was formed which quickly overloaded the motor. By running a vacuum, heat can be evacuated from the mill, thereby allowing for higher throughput operating conditions.

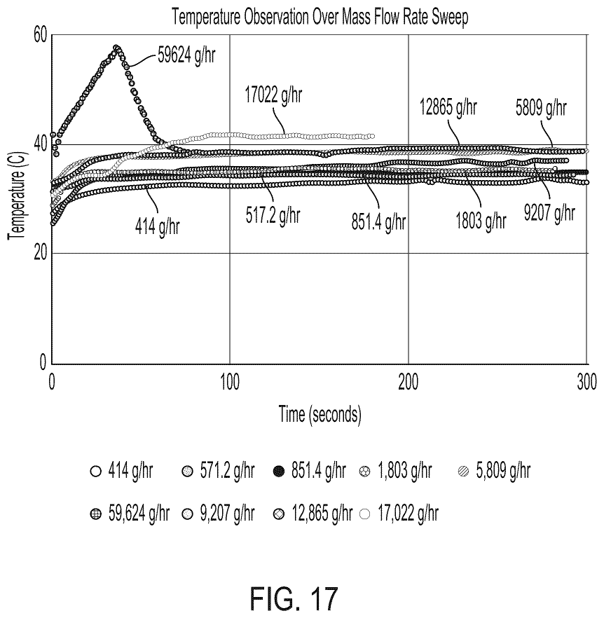

The information from the previous experiment was used to test the maximum throughput of the rotor mill before failure. In the following example, PET pellets were milled in a centrifugal mill and evacuated to an external collecting vessel by a vacuum powered cyclone. The material was fed by a vibratory feeder (Retsch DR100). The maximum throughput was defined as the input flow rate that caused rotor mill failure. As seen in FIG. 17, at −60 kg/hr, the rotor mill motor quickly overloaded. The maximum throughput therefore was in the range of 20-60 kg/hr.

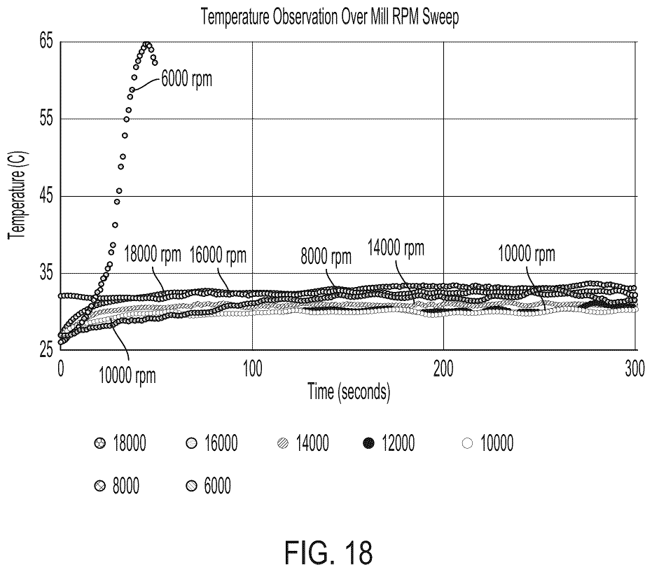

A similar experiment was conducted on the rotor mills revolutions per min (RPM) control capability. The rotor mill has a range of 6000-18000 RPM. Flow rate was controlled using the vibratory feeder at 390 g/hr. Temperature was once again measured within the receiving bowl of Retsch. For the RPM range of 6000-16000, temperature remained relatively consistent and steady. As indicated in FIG. 18, the mill rpm is limited to 8000 rpm, as there is a stall that occurs at 6000 rpm. The operating conditions for 390 g/hr therefore lies between 8000-18000 RPM.

### Sections 6-9:

It was discovered that by controlling milling conditions of post-consumer PET flakes (rPET) one can significantly improve the speed and yield of depolymerization by enzymes. It was demonstrated that one can control the physical state of milled particles by adjusting milling conditions based on a variety of parameters, namely the mean residence time within the mill and the steady-state temperature of the mill to optimize for enzymatic depolymerization. It was discovered that a balance need to be achieved between mean residence time and cooling rate within the mill to develop a practical continuous process for pre-processing rPET for enzymatic depolymerization.

In this section, for a given mill, a method to optimize material throughput by measuring mean residence time to achieve particles with improved speed and yield of depolymerization by enzymes is described. The technique allows one to avoid pre-processing of the rPET material by classical methods involving high-temperature extrusion and fast cooling.

### Section 6:

In this section, the concept of mean residence time in the milling process will be shown as a driving force for preparing polymers for enzymatic degradation. The mean residence time can be used to predict mill performance for both throughput and quality of milled particles for enzymatic depolymerization. Surprisingly, it was demonstrated that by changing conditions of milling of PET pellets, milled particles could become less recalcitrant to enzyme catalyzed depolymerization. Control of milling conditions led to both a faster depolymerization and to a higher yield, as measured by volume of NaOH dosed during the reaction. A key parameter to be considered was the amount of time the material spends within the mill, or the material's residence time. Residence time can be defined as the time for the mill to return to idle power draw after a set mass of material is quickly introduced to the mill. The mean residence time can be defined as the average of a minimum of three trials for a given pulse mass.

### Example 25

The free volume available in the mill (Retsch ZM200) can directly influence the time material will spend in milling. In this example, two different ring sieve sizes were used for 0.2 g pulse tests of rPET flake (PolyQuest Recycling, Polyethylene Terephthalic Flake—Post Consumer Waste). The ring sieve sizes are characterized as Short Ring Sieve (RETSCH 36470248) (10 cm diameter) and Distance Ring Sieve (RETSCH 36470257) (11.3 cm diameter), both ring sieves having a mesh size of 0.5 mm and Conidur style holes. By running the mill in idle conditions at 18000 rpm, i.e. no material being presented to the mill, the power draw was maintained at a baseline of 700 watts. A pulse mass of 0.2 grams was added to the mill quickly manually, thereby causing the power draw of the mill to spike to an average value of 1,878 watts for the Short Ring Sieve and 1,197 watts for the Distance Ring Sieve before returning to idle condition. Repeating this procedure a minimum of three times and averaging the time before returning to the idle conditions results in a given pulse mass's mean residence time.

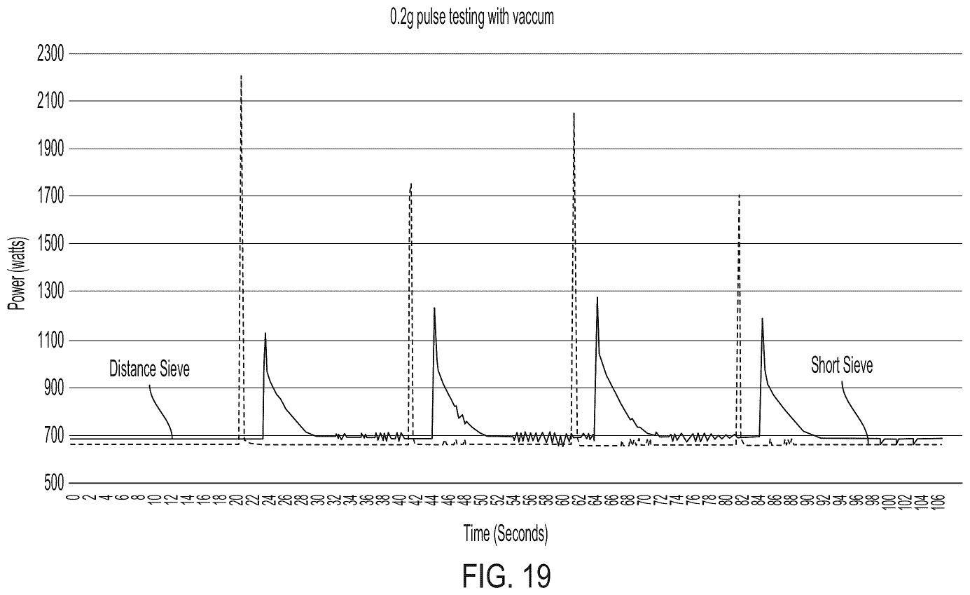

As shown in FIG. 19, the distance ring sieve had a mean residence time, measured in triplicate, of 7.67±1.92 seconds while the short ring sieve had a mean residence time, measured in triplicate, of 0.71±0.06 seconds for a 0.2 gram pulse mass.

### Example 26

The pulse tests can be used to determine the theoretical limit for mill throughput under a specific set of conditions. In this example, pulse mass was varied in the range of 0.1-6 gram and the mean residence time was measured for each pulse mass. One can then calculate the fully loaded duty-cycle of the mill, and thus the apparent mass flow, by the following equation:

$$Apparent Mass Flow ( \frac{k g}{h r} ) = \frac{Pulse mass [ kg ]}{Mean Residence Time [ hr ]} \qquad Equation 2$$

For example, by using 0.2 gram pulses and the short ring sieve, a mean residence time of 0.71 seconds was measured, which results in an apparent mass flow of 1.01 kg/hr.

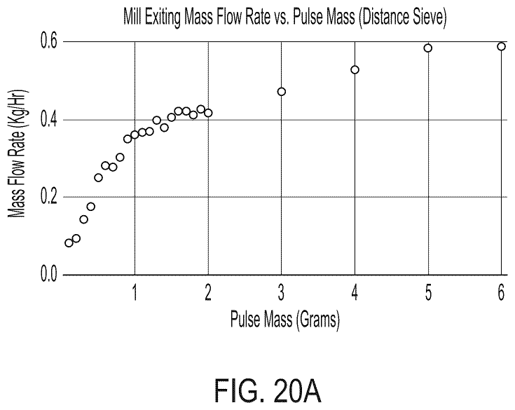

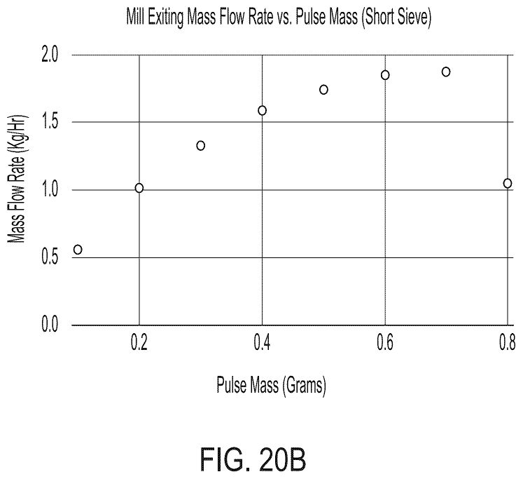

FIGS. 20A-20B illustrate the result of repeating this procedure for different pulse masses using both the distance and short ring sieves. As shown, the plateau of this curve elucidates a good starting point for average throughput a mill can tolerate before failure. For the distance ring sieve (FIG. 20A), the throughput limit lies around 0.4-0.6 kg/hr while for the short ring sieve (FIG. 20B), the throughput limit is estimated to be around 1.7-2 kg/hr.

### Example 27

In this example, experiments were conducted at the expected throughput limit estimated in Example 26. A Loss-in-Weight feeder (Movacolor MCBalance Gravimetric Single Screw Feeder) was used to feed at different flow rates for both the short and distance ring sieves. For the short sieve, the throughput limit was estimated to be 2 kg/hr from Example 26. The milling process was run for one hour with ring sieve temperature being measured by an insulated thermocouple (Omega 5TC-GG-K-30-36 glass). The temperature of the short ring sieve reached a steady-state at 49° C. The mass flow rate was increased to 2.25 kg/hr for another one-hour test. In this test, after 6.5 minutes, the temperature of the sieve began climbing to above 55° C. before a film was formed inside the ring sieve from melted material, thereby stalling the motor and halting the experiment.

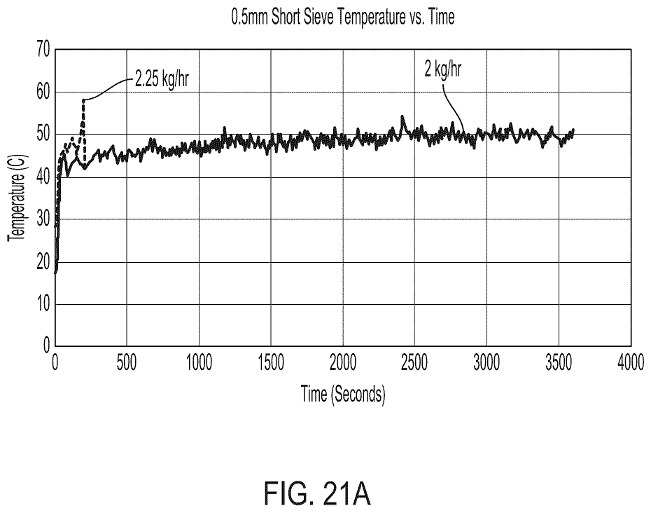

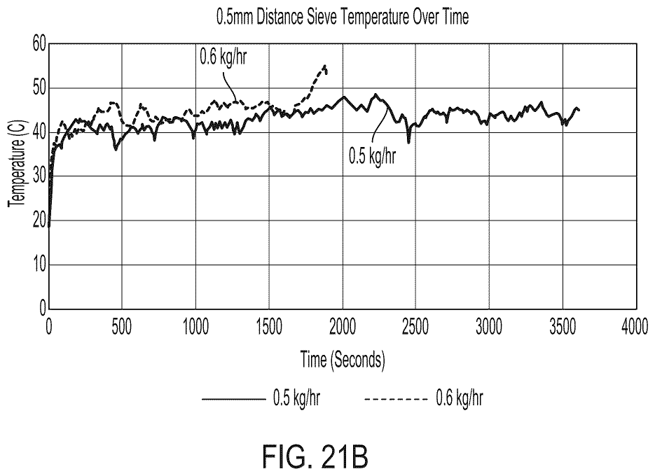

The pulse tests accurately predicted the material throughput limit of the mill. The distance ring sieve was trialed next at 18000 RPM with a flow rate of 0.5 kg/hr, as predicted from Example 26. The temperature of the distance ring sieve reached a steady-state at 44° C. The mass flow rate was increased to 0.6 kg/hr for another one-hour test. Similarly, to the short ring sieve, after 31.5 minutes the temperature of the distance ring sieve began climbing to above 55° C. before a film was formed inside the ring sieve from melted material, thereby stalling the motor and halting the trial. The temperature of the ring sieves was measured for each of these experiments and shown in FIG. 21A and FIG. 21B below.

### Example 28

In this example, each of the milled samples from Example 27, e.g., milled sample resulting from the flow rate of 2 kg/hr using the short ring sieve and milled sample resulting from the flow rate of 0.5 kg/hr using the distance ring sieve, was fractionated and enzymatically depolymerized, according to some embodiments.

### Sieving

Each of the milled samples from Example 27, namely, milled sample resulting from the flow rate of 2 kg/hr using the short ring sieve and milled sample resulting from the flow rate of 0.5 kg/hr using the distance ring sieve, was fractionated by sieving using an analytical sieve shaker (Retsch, AS200) operating at an amplitude of about 1 mm/“g” (30 minutes of total shaking). Fractionation was performed using stainless steel test sieves (Retsch) with a diameter of 200 mm and mesh sizes of: 500 m, 400 m, 300 m, 200 m, 100 m, and 50 m.

### Reaction of PET Fraction

For each of the milled samples, the sieved fraction having sizes between 200-300 m was enzymatically depolymerized in a bioreactor using Novozyme HiC 51032 (LOT L01332211, purchased from Strem Chemicals). The conditions of the reaction were 65° C., 10% solid loading of PET (10 g in 90 mL), 100 millimolar potassium phosphate buffer at pH 8.0, and 5 mL of enzyme solution as received. The pH of the reaction was controlled using a Raspberry Pi controlled system that was employed to dose 6 molar sodium hydroxide to maintain a pH of 8.0.

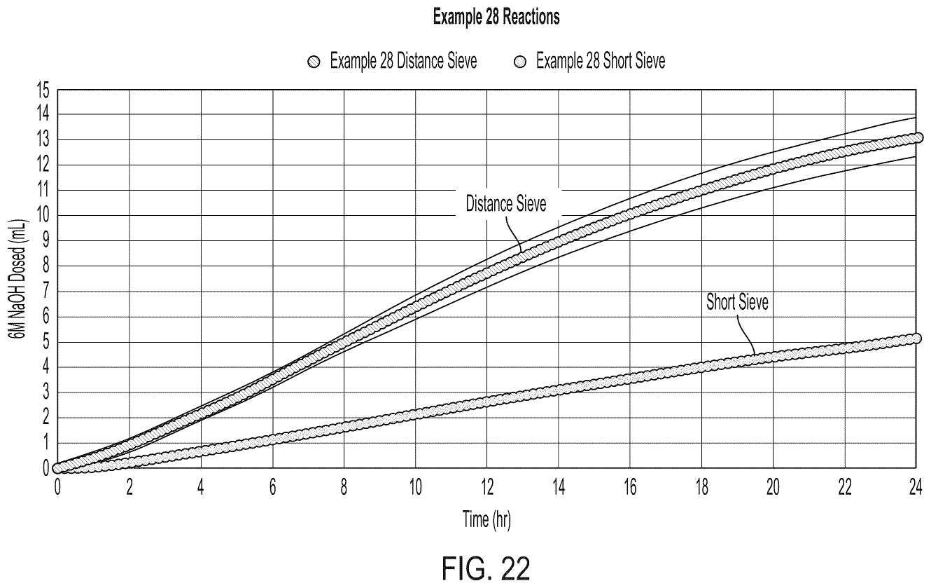

The reaction progression and kinetics can be understood by the dosing rate of the sodium hydroxide, as shown in FIG. 22. The equation used to correlate sodium hydroxide dosed to reaction completion is shown below:

$$\% Completion = \frac{\begin{matrix}Volume Base Dosed ( L ) * \\ Molarity ( \frac{Moles}{L} ) * 166.13 ( \frac{g}{mol} )\end{matrix}}{2 * Theoretical Yield} \qquad Equation 3$$

For 10 grams of PET, the theoretical yield of Terephthalic Acid was calculated to be 8.64 grams. For the sieved fraction of milled sample employing the distance ring sieve, reactions were dosed at 4.87±0.30 mL of 6M sodium hydroxide after 22.5 hours, which gave rise to a reaction completeness of 28.1±1.7%. For the sieved fraction of milled sample employing the short ring sieve, reactions were dosed at 12.92±0.29 mL of 6M sodium hydroxide after 22.5 hours, which gave rise to a reaction completeness of 74.5±1.7%. Based on this experiment and the throughput limits described in Example 27, only the short ring sieve was considered for future milling experiments.

### Example 29

Each of the samples from Example 27, namely, milled sample resulting from the flow rate of 2 kg/hr using the short ring sieve (i.e., “short ring sieve 2 kg/hr sample”) and milled sample resulting from the flow rate of 0.5 kg/hr using the distance ring sieve (i.e., “distance ring sieve 0.2 kg/hr sample”), were analyzed using Differential Scanning Calorimetry (TA DSC25) to determine the crystallinity degree (CD) of material.

### DSC Heating Scans

The crystallinity degree determined from DSC first heating scan (CD) is defined by the following expression:

$$CD = \frac{( Δ {H}_{melt} - Δ {H}_{cold crystallization} )}{Δ {H}_{melt}^{°}} \cdot 100 \qquad Equation 4$$

where ΔHₘₑₗₜ is the normalized enthalpy of melting of PET, ΔH_{crystallization} is the normalized enthalpy of crystallization of PET, and ΔH°ₘₑₗₜ is the normalized enthalpy of melting of a 100% crystalline polymer and/or PC/IPM at the melting temperature. ΔH°ₘₑₗₜ for PET is 140.1 J/g.

DSC first heating scans of PET samples were obtained using a calorimeter (TA, DSC 25). 3-10 mg of PET extrudate sample were cut and introduced in a capsule (TA, Standard Pan 900786.901 and Standard Lid 900779.901). The first heating scan was collected using the following protocols: 1) Equilibrate temperature from room temperature to 0° C.; 2) Isothermal step (0° C.) for 1 min; and 3) Heating step from 0° C. to 300° C. at a heating rate of 10° C./min. From the normalized heat flow curve versus temperature, the CD was calculated as defined in Eq. 1 using the TRIOS software version 5.6.0.87. The integration of the crystallization exothermic peak which is around 110-140° C. and the melting endothermic peak which is around 230-250° C. were performed from 100-170° C. and 210-270° C. using a straight baseline between them to get ΔHₘₑₗₜ and ΔH_{crystallization}. The glass transition temperature (T_{g}) (midpoint), crystallization temperature (T_{cryst}) (mean and onset) and melting temperature (T_{melting}) (mean and onset) were determined using the TRIOS software version 5.6.0.87.

The post-consumer PET flakes (rPET) purchased from PolyQuest is a non-homogenous material having different sizes (thickness), mixed colors as it refers to ground washed bottle flakes from beverage, food, and non-food packaging. Since the different parts of the PET plastic bottles (neck, shoulder, body, etc.) were not homogeneous, it was expected that the crystallinity degree of rPET flakes would vary from flake to flake. For this reason and to have a better characterization of the average CD of the staring material, DSC experiments were performed on different flakes and on cryo-milled rPET.

The following example illustrates the variability of CD of rPET flakes: A thick flake (thickness of 1.5 mm) from rPET was cut with a scissor and characterized by DSC first heating scan. The CD of the thick flake sample was 11%. A thin flake (thickness of 0.2-0.4 mm) was randomly picked from the rPET sample and cut with a scissor. The cuts of rPET thin flake were characterized by DSC. The CD of the thin flake was 27%.

rPET (50 g) were placed in a beaker and pre-cooled in liquid nitrogen for 1 min. Then the pre-cooled rPET flakes were transferred to a Moulinex grinder (50 g, 180 W) and milled for 1 min. The cooling step and milling step were repeated 4 times to give a total milling time of 4 min. The obtained fragments were characterized by DSC (by triplicate) to estimate an average crystallinity degree of rPET sample. The CD of the fraction was (24±2)%.

The milled samples from Example 27 were then compared using the DSC analysis outlined above. The global, meaning unsieved (unfractionated) powder of the short ring sieve 2 kg/hr sample was measured in triplicate, thereby resulting in a CD of 7.1±0.8%. The global ring distance sieve 0.5 kg/hr sample was measured in triplicate resulting in a CD of 22.0±0.7%. The short ring sieve powder exhibited a reduced CD compared to the starting material which was not true of the distance ring sieve. Without wishing to be bound by any particular theory, it is hypothesized that (additional) decrease of recalcitrance of milled crystallizable polymers or copolymers and PC/IPM containing crystallizable polymers or copolymers may, at least in part, be associated with a decrease in the degree of crystallinity of the crystallizable polymers or copolymers during the milling process. The shorter residence times of the short sieve allow for both an increased throughput and desirable qualities in the powder produced, that is small particle sizes and more amorphous plastic.

### Section 7:

In this section, the classical method of producing enzymatically digestible plastic powder is compared to the milling process described in Section 6. The typical method consists of extruding PET followed by a fast-cooling process in water, before pelletizing the extrudate and finally milling into a powder to increase the surface area.

### Comparative Example 30

rPET Flake (PolyQuest Recycling, Polyethylene Terephthalic Flake—Post Consumer Waste) was extruded as received using a Leistritz ZSE 27 MAXX twin-screw extruder at a melt temperature of 280° C. at 64 kg/hr. The extrudate was quenched by extruding into a room temperature water bath before being pelletized. The extruded PET pellet was measured in triplicate using the DSC protocol from Example 29 yielding a CD of 8.12±0.43%.

The amorphous pellets were milled using the Retsch ZM200 using the short ring sieve (10 mm diameter) at 18,000 RPM with full vacuum. The feed rate of pellets into the mill was controlled using a Movacolor MCBalance Gravimetric Single Screw Feeder at 0.25 kg/hr. Any feed rate higher than 0.25 kg/hr would result in gradual heat up of the short ring sieve and a film formation that interrupted milling. The milled PET powder was measured in triplicate using the DSC protocol from Example 29 yielding a CD of 9.1±0.2%.

### Example 31

rPET Flake (PolyQuest Recycling, Polyethylene Terephthalic Flake—Post Consumer Waste) was milled as received with the Retsch ZM200 using the short ring sieve (10 mm diameter) at 18,000 RPM with full vacuum. The feed rate of flakes into the mill was controlled using a Movacolor MCBalance Gravimetric Single Screw Feeder at 0.25 kg/hr to match the feed rate of Comparative Example 30. The maximum feed rate of the flake, as shown in Example 17, could reach up to 2 kg/hr, nearly an order of magnitude greater than that of the pellets. The increased flow rate can be advantageous when considering scaling up this technology. The milled rPET powder was measured in triplicate using the DSC protocol from Example 29 yielding a CD of 7.2±0.6%.

### Example 32

PET Tray with PE, EVA (Clamshell containers, Change Plastic for Good) was milled as received with the Retsch ZM200 using the short ring sieve (10 mm diameter) at 18,000 RPM with full vacuum. The feed rate of 2-3 mm tray into the mill was controlled using a Movacolor MCBalance Gravimetric Single Screw Feeder at 0.25 kg/hr to match the feed rate of Comparative Example 30. The maximum feed rate of the flake could reach up to 0.5 kg/hr. The PET Tray material before processing was measured in triplicate using the DSC protocol from Example 29 yielding a CD of 31.1±4.6%. The milled powder was measured in triplicate using the DSC protocol from Example 29 yielding a CD of 7.9±0.6%.

### Example 33

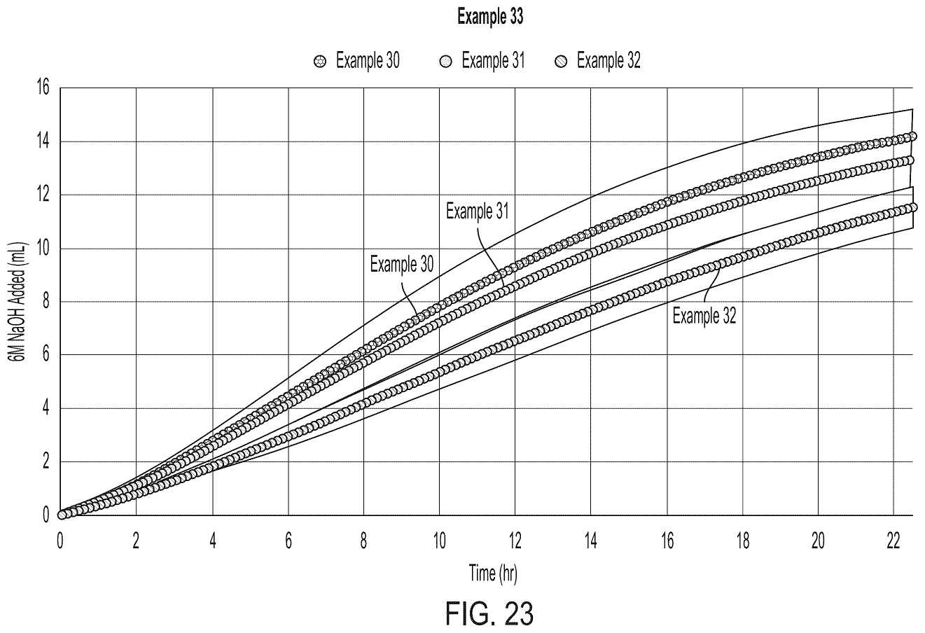

The material from Examples 30, 31, and 32 were sieved according to the protocol in Example 28 to obtain a powder with particle size distribution between 200-300 microns. Each sample was enzymatically depolymerized in a bioreactor using the conditions and protocol outlined in Example 28. For the sample from Example 30 (i.e., the milled pellets sample), reactions were dosed at 14.23±0.94 mL of 6M sodium hydroxide after 22.5 hours, which gave rise to a reaction completeness of 82.1±5.4%. For the sample from Example 31 (i.e., the milled flake sample), reactions were dosed at 13.32±1.06 mL of 6M sodium hydroxide after 22.5 hours, which gave rise to a reaction completeness of 76.8±6.1%. For the sample from Example 32 (i.e., the milled tray sample), reactions were dosed at 11.53±0.69 mL of 6M sodium hydroxide after 22.5 hours, which gave rise to a reaction completeness of 66.5±3.9%. The rate of NaOH dosing is shown in FIG. 23. Reaction completeness was calculated in the same way as in Example 28. With the reactions yielding similar completeness, Example 31 appears to be a preferable process compared to Example 30 due to the increased throughput shown in Example 27 and simplicity of the process. Example 32 shows that the same process employed in Example 30 can be used on varying types of PET.

### Section 8:

In this section, the milling process described in Section 6 is demonstrated to be adaptable to certain mills and less compatible with others. For example, with an attrition mill, the mechanical process variables are the gap between the rotors, material feed rate into the mill, air flow through the mill, and the RPM of the rotors. By tuning these variables, a process can be developed to produce material with enhanced capability for enzymatic depolymerization.

### Example 34

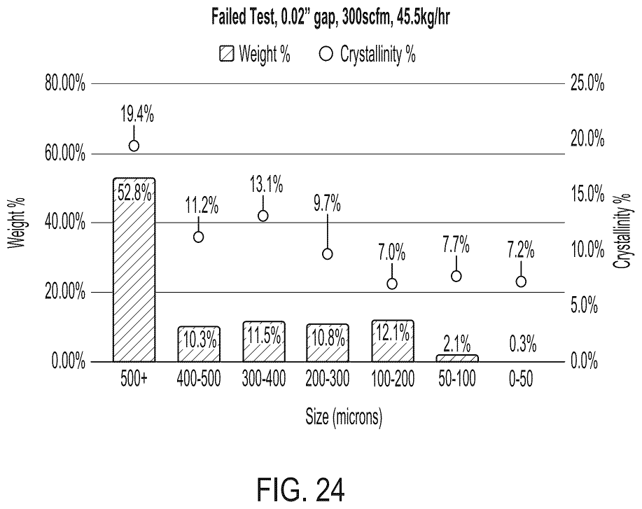

rPET Flake (PolyQuest Recycling, Polyethylene Terephthalic Flake—Post Consumer Waste) was conveyed with a volumetric feeder (Coperion K-Tron Volumetric Single Screw), calibrated to 45.5 kg/hr, feeding into the inlet of the mill (The Mikro V-UMP 15 hp) for 4.6 minutes. The attrition mill was set to a 0.020″ gap and a 300 scfm blower speed. The rotors used were the coarse serrated teeth rotors. The mill outlet temperature was measured at 60° C. Due to the conditions, heat was increasing within the mill due to the energy being imparted on the flakes. Similar to the Retsch ZM200, the heat generated was enough to melt the rPET fed into the mill and began agglomerating until the agglomerated mass blocked the mill exit. Too high of a material throughput increases the mill temperature and results in this failure mode. The powder generated from this trial were sieved and analyzed using a DSC for the CD as described in Examples 28 and 29. The particle size distribution and CD for the sieved fractions are shown in FIG. 24.

### Example 35

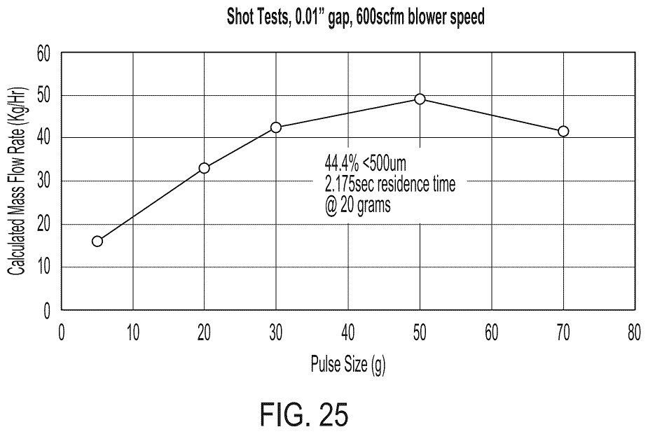

Pulse tests were performed to understand the mean residence time and maximum throughput of the mill. For different gap sizes and air flow rates, outlined in Table 3, pulse tests were conducted using a range of pulse masses between 5 and 70 grams per pulse. A characteristic analysis similar to Example 26 was conducted for each condition, as shown in FIG. 25. The key metrics measured were the mean residence time for the pulses, the percent of material smaller than 500 μm, and the estimated throughput on the mill at that given condition.

TABLE 3
Conditions tested on Hosokawa V-UMP 15 hp

| Test | Gap Size (inches) | Air Flow (SCFM) |
|---|---|---|
| 1 | 0.001 | 400 |
| 2 | 0.001 | 600 |
| 3 | 0.0015 | 400 |
| 4 | 0.0015 | 600 |
| 5 | 0.002 | 400 |
| 6 | 0.002 | 600 |

### Example 36

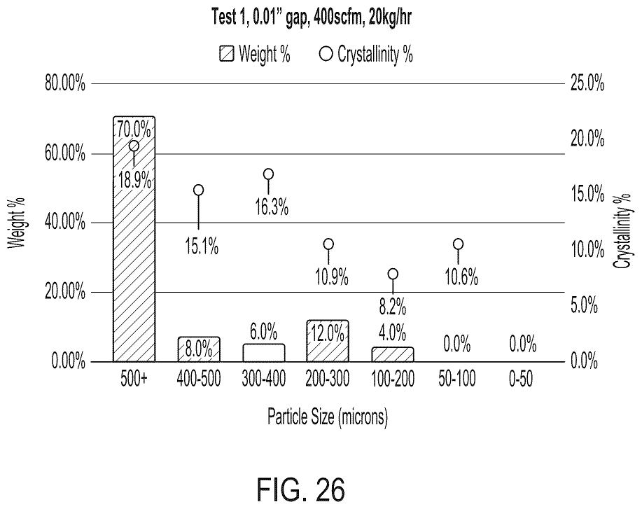

Using the metrics from the pulse tests on the process space in Example 35, steady-state conditions were explored rather than pulse tests, namely for the conditions in Example 35 Test 1. rPET Flakes (PolyQuest Recycling, Polyethylene Terephthalic Flake—Post Consumer Waste) were fed at a rate of 20 kg/hr using the conditions in Example 35 Test 1. The powder from this trial was sieved according to the protocol in Example 28 and measured by DSC according to the protocols in Example 29. The global CD of the material out of the mill was measured to be 17.07±1.85%. For every test condition in Table 1, the same steady-state conditions of 20 kg/hr were tested. A sample of each condition was sieved and measured by DSC using the same protocols in Example 28. The results are shown in FIG. 26. The trend of higher CD in larger particles held for the 20 kg/hr trials at each test condition in Table 3. Test 1 was privileged over the other tests for its low residence time and particle size distribution.

### Example 37

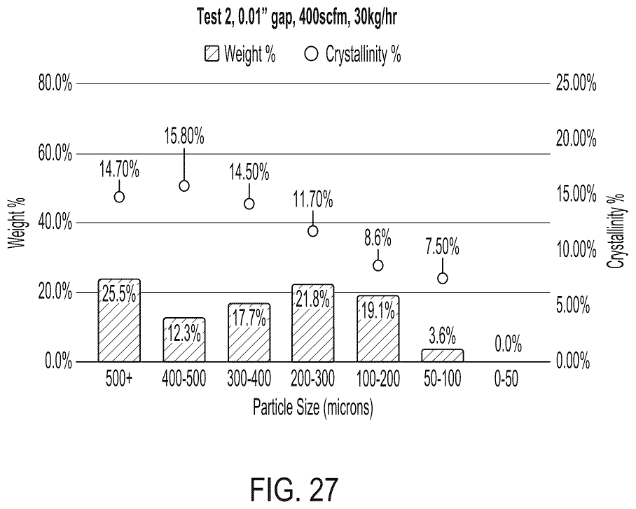

The conditions from Table 3—Test 1 were repeated, however the flow rate was increased to 30 kg/hr. The powder from this trial was sieved according to the protocol in Example 28 and measured by DSC according to the protocols in Example 29. The particle size distribution and CD for the sieved fractions are shown in FIG. 27. The global CD of the material out of the mill was measured to be 13.04±0.43%. In this example, even the particles larger than 500 micron exhibited a decrease in CD after milling. With an attrition mill, it was observed that a balance of throughput and particle size distribution of resultant powder is necessary to achieve ideal conditions for PET suitable for enzymatic depolymerization. The narrowing of the large process space to achieve amorphous particles, desirable particle size distribution, and practical operating conditions were found through using the mean residence time and pulse tests.

### Example 38

The material from Example 37 were sieved to obtain a powder with particle size distribution between 200-300 microns. The powder was enzymatically depolymerized using the reaction conditions expressed in Example 26. The reaction progression and kinetics can be understood by the dosing rate of the sodium hydroxide. The reaction dosed 12.87±0.78 mL of 6M sodium hydroxide after 22.5 hours, which corresponds to a reaction completeness of 74.2±4.5%. The material milled under specific conditions determined by pulse tests and a knowledge of the milling process showed similar reaction kinetics and yield to material prepared in the classical manner from Example 30 and Example 32. The new method of preparing material for enzymatic depolymerization is preferable to classical methods due to its increased simplicity and lower cost.

### Example 39

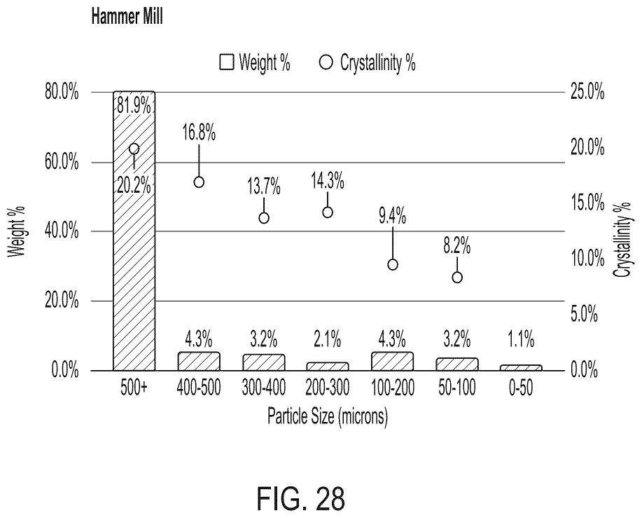

The hammer mill is another mill designed for fine size reduction of materials. With a hammer mill, the mechanical process variables can include the style of hammer, the retaining screen style and hole size, material feed rate into the mill, air flow through the mill, and the RPM of the hammers. A hammer mill (The Mikro Pulverizer® Hammer & Screen Mill, Hosokawa Micron Powder Systems) was set up with a 0.093″ hole size screen, blower speed of 400 scfm, rotor speed of 9,600 rpm. Using a 10 gram pulse mass, the mean residence time of the hammer mill was 1.625 seconds. The comparable mean residence time to the attrition mill indicates that the hammer mill may be a potentially viable mill for producing enzymatically favored particles. The hammer mill conditions were kept the same for a material feed rate of 12 kg/hr. The powder generated from this trial was sieved and analyzed by DSC as in Example 28 and shown in FIG. 28. The global CD of the material out of the mill was measured in triplicate to be 19.6±2.9%. Similar to Example 36, the throughput through the mill at adequate particle sizes was insufficient, with more than 80% of the material being larger than the target 500 microns. Although this test yielded undesirable particle size distributions, the short mean residence time indicates the hammer mill may have a process space in which desirable particles can be generated.

### Section 9:

The following Comparative Example illustrates that micronization of rPET for a long residence time by ball milling occurs with no significant change in the degree of crystallinity of the sample.

### Comparative Example 40

50 g of rPET flakes (PolyQuest Recycling, Polyethylene Terephthalic Flake—Post Consumer Waste) were placed in a beaker and pre-cooled in liquid nitrogen for 1 min. Then the pre-cooled rPET flakes were transferred to a Moulinex grinder (50 g, 180 W) and milled for 1 min. The cooling step and milling step were repeated 4 times to give a total milling time of 4 min.

### Comparative Example 41

5 g of the obtained milled material described in the Comparative Example 40 were further milled using a ball mill (Retsch Cryogenic ball mill, ref #20.749.0001), zirconia (ZrO₂) jar of 25 mL and the following zirconia (ZrO₂) grinding balls: 1 ball of diameter 20 mm and 2 balls of diameter 9 mm.

Ball milling was performed using a 2 min milling cycle at 30 Hz, followed by a 5 min pause at 5 Hz. Ball milling was performed starting from room temperature (30° C.) and the 5 min pause step was used to avoid heat buildup. The sequence of 2 min milling cycle followed by 5 min pause was repeated up to 60 min of total milling (30 milling cycles; total residence time: 60 min). Milling was stopped and the sample was left inside the jar for temperature equilibration at room temperature. Then, the sample was further milled starting from room temperature (30° C.) to a total milling time of 120 min (60 milling cycles; total residence time: 120 min), following the sequence of 2 min milling cycles at 30 Hz, followed by a 5 min pause at 5 Hz. Then the jar was opened and the temperature of the sample was measured with a thermocouple to be 47° C.

### Characterization:

The crystallinity degree was determined from DSC first heating as defined in the Example 29.

Differential Scanning Calorimetry (DSC) first heating scan of rPET samples was obtained using a calorimeter (TA, discovery Q200). 10 mg of PET were introduced in a capsule (TA, Tzero Pan T 220228 and Tzero Hermetic Lid T 220315). The first heating scan was collected following the sequence: 1) Equilibrate temperature from room temperature to 0° C.; 2) Isothermal step (0° C.) for 1 min; 3) Heating step from 0° C. to 300° C. at a heating rate of 10° C./min. From the normalized heat flow curve vs temperature, the degree of crystallinity was calculated as defined in the Example 29 using the TRIOS software version v3.1.5.3696. The integration of the cold crystallization exothermic peak at around (115-135) ° C. and the melting endothermic peak at around 250° C. was performed from visually determined respective starting points to end points using a straight baseline between them to get ΔH_{cold crystallization} and ΔHₘₑₗₜ, respectively.

The crystallinity degree is of (28±2) % for the rPET obtained by Moulinex grinder described in the Comparative Example 40 and of (30±2) % for the rPET milled by ball milling as described in the Comparative Example 41.

The particle size distribution of rPET milled by ball milling as described in the Comparative Example 41 was measured by Laser Diffraction using a Microtrac Sync in the wet mode.

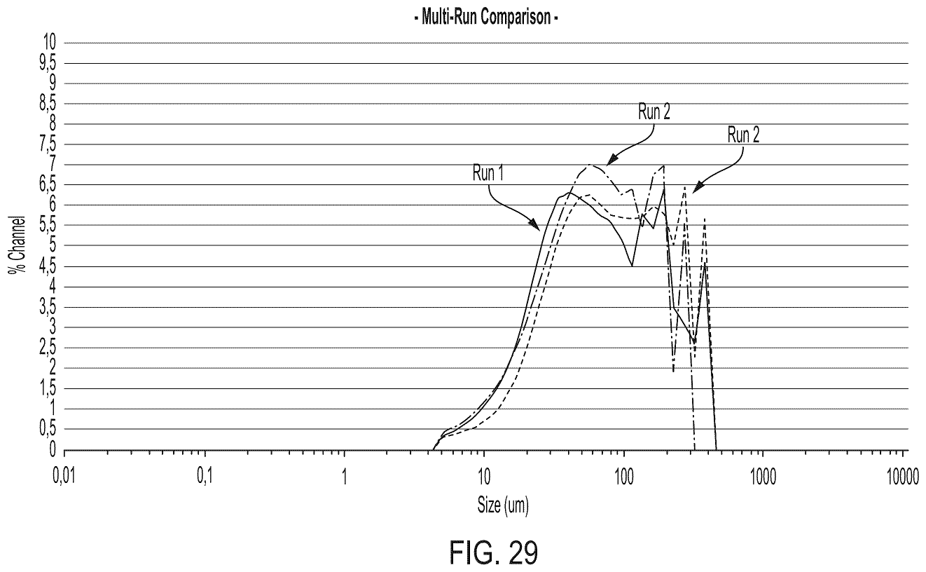

100 mg of the micronized rPET obtained by ball milling as described in the Comparative Example 41 were dispersed by mechanical stirring in 10 mL of distilled water. Dispersion of particles was loaded into the Microtrac Sync to perform the particle size analysis in triplicates (each measurement time was 30 seconds), as shown in FIG. 29.

TABLE 4
Summary of results of particle size distribution of rPET milled sample
obtained by ball milling as described in the Comparative Example 41:
Percentiles

| % Tile | Size(μm) |
|---|---|
| 10 | 20.40 |
| 50 | 71.79 |
| 90 | 249.5 |

### Example 42

This example illustrates that milling stress or flow-induced oriented/crystallized polymeric materials is particularly advantageous to achieve a high enzymatic depolymerization rate and yield. Examples of stress-induced oriented/crystallized materials are biaxially oriented PET films, PET bottle flakes, PET bottles, PET preforms to make bottles, PET trays, among others. In this example stress-induced oriented/crystallized materials are illustrated with a biaxially oriented PET polyester film.

### Example 42.1: Milling PET Obtained by Melt Extrusion Followed by Cooling at Room Temperature

In one milling condition (42.1), a biaxially oriented PET polyester film (Toyobo ester E5001, Toyobo Co., LTD.) with a thickness of 250 μm was cut with scissors into flakes of sizes around 0.5 cm×0.5 cm to around 0.8 cm to 1.0 cm. Such biaxially oriented PET polyester flakes (12 g) and an antioxidant (0.1 wt. %, Irganox 1010, Sigma) were fed into a hot conical twin screw compounder (DSM, Xplore, 15 cm³ capacity) equipped with co-rotating conical screws, recirculation channel to control the residence time and a circle die with diameter of 3.0 mm allowing for extrusion of the material from the compounder. The feeding/mixing/extrusion were performed under circulation of nitrogen, with a barrel temperature profile as follows: top position (270° C.), middle position (270° C.), and exit position (280° C.). The speed of rotation of the screws was 60 RPM. The extruder was filled in around 1 min. After feeding the extruder, the compound was mixed. At a mixing time of 1.5 min the sample was withdrawn directly through the die, extruded and kept at room temperature. The extrudate was cut with a ply into pieces (2-5) mm. 10 g of extrudate cuts were milled with a centrifugal mill (Retsch ZM200) at 14000 RPM, using a sieve with a mesh size of 0.5 mm and a diameter of 10 cm (small teeth-to-sieve gap). The rotor having 12 rotor teeth arranged along the external circumference of the rotor (diameter 9.8 cm). Extrudate cuts were fed into the mill at room temperature at an average feed rate of 3.3 g/min during 3 min. The temperature of the sieve was monitored during the milling time. The maximum temperature was 55° C. and the crystallinity degree of the micronized PET was 31%.

### Example 42.2: Milling PET Obtained by Classical Amorphisation Followed by Fast Cooling

In one milling condition (42.2), a biaxially oriented PET polyester film (Toyobo ester E5001, Toyobo Co., LTD.) with a thickness of 250 μm was cut with scissors into flakes of sizes around 0.5 cm×0.5 cm to around 0.8 cm to 1.0 cm. Such biaxially oriented PET polyester flakes (12 g) and an antioxidant (0.1 wt. %, Irganox 1010, Sigma) were fed into a hot conical twin screw compounder (DSM, Xplore, 15 cm³ capacity) equipped with co-rotating conical screws, recirculation channel to control the residence time and a circle die with diameter of 3.0 mm allowing for extrusion of the material from the compounder. The feeding/mixing/extrusion were performed under circulation of nitrogen, with a barrel temperature profile as follows: top position (270° C.), middle position (270° C.), and exit position (280° C.). The speed of rotation of the screws was 60 RPM. The extruder was filled in around 1 min. After feeding the extruder, the compound was mixed. At a mixing time of 1.5 min the sample was withdrawn directly through the die, extruded into an ice/water bath kept at 5° C. The extrudate was cut with a ply into pieces (2-5) mm. 11 g of extrudate cuts were milled with a centrifugal mill (Retsch ZM200) at 14000 RPM, using a sieve with a mesh size of 0.5 mm and a diameter of 10 cm (small teeth-to-sieve gap). The rotor having 12 rotor teeth arranged along the external circumference of the rotor (diameter 9.8 cm). Extrudate cuts were fed into the mill at room temperature at an average feed rate of 3.7 g/min during 3 min. The temperature of the sieve was monitored during the milling time. The maximum temperature was 56° C. and the crystallinity degree of the micronized PET was 17%.

### Example 42.3: Milling Biaxially Oriented PET Film

In one milling condition (42.3), a biaxially oriented PET polyester film (Toyobo ester E5001, Toyobo Co., LTD.) with a thickness of 250 μm was cut with scissors into flakes of sizes around 0.5 cm×0.5 cm to around 0.8 cm to 1.0 cm. Such biaxially oriented PET polyester flakes (47 g) were milled with a centrifugal mill (Retsch ZM200) at 14000 RPM, using a sieve with a mesh size of 0.5 mm and a diameter of 10 cm (small teeth-to-sieve gap). The rotor having 12 rotor teeth arranged along the external circumference of the rotor (diameter 9.8 cm). Biaxially oriented PET polyester flakes were fed into the mill at room temperature at an average feed rate of 4 g/min during 11.75 min. The temperature of the sieve was monitored during the milling time. The maximum temperature was 85° C. and the crystallinity degree of the micronized PET was 6%.

### Example 42.4: Milling Non-Oriented PET Pellets

In one milling condition (42.4), PET pellets (50 g) (RAMAPET N1(S) supplied by Indorama Ventures) were milled with a centrifugal mill (Retsch ZM200) at 14000 RPM, using a sieve with a mesh size of 0.5 mm and a diameter of 10 cm (small teeth-to-sieve gap). The rotor having 12 rotor teeth arranged along the external circumference of the rotor (diameter 9.8 cm). Indorama PET pellets were fed into the mill at room temperature at an average feed rate of 5 g/min during 10 min. The temperature of the sieve was monitored during the milling time. The maximum temperature was 63° C. and the crystallinity degree of the micronized PET was 36%.

### Fractionation of Milled Samples by Size:

Each of the milled samples described in Example 42.1, Example 42.2, Example 42.3 and Example 42.4 were fractionated by sieving using an analytical sieve shaker (Retsch, AS200) operating at an amplitude of 0.7 mm for 10 minutes. Fractionation was performed using stainless steel test sieves (Retsch) with a diameter of 100 mm and mesh sizes of: 300 μm and 150 μm. The milled fraction retained by the 150 μm mesh (containing the fraction of sizes between 150 μm and 300 μm) was used for the enzymatic degradation characterization.

### Absorbance Method to Measure the Enzymatic Depolymerization.

The following paragraph describes the method to determine enzymatic depolymerization reaction yield at 24 h of polymeric materials from absorbance measurement. Depolymerization reaction yield measured by terephthalic acid (TPA) equivalent production and enzymatic depolymerization reaction time are referred below as “reaction yield” and “reaction time”, respectively.

### Calibration Curve for Determination of Terephthalic Acid (TPA) Equivalent in the Reaction Bath

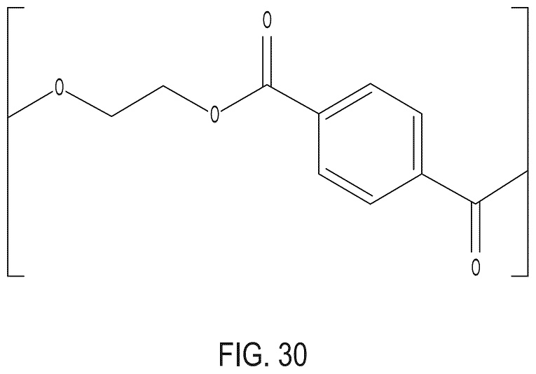

The repeating units of PET contains terephthalate units as presented in FIG. 30.

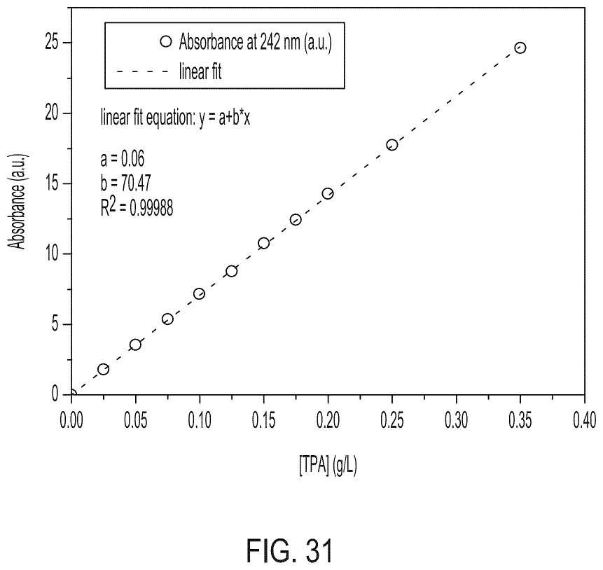

Upon enzymatic depolymerization of PET, TPA and/or soluble low molecular weight molecules such as mono(2-hydroxyethyl) terephthalate and bis(2-hydroxyethyl) terephthalate for example, are released into the solution as depolymerization products. TPA has a maximal absorption band in UV-visible spectrum at 242 nm. All other soluble molecules containing esters of terephthalic acid contribute to the absorbance signal as well. UV-Visible spectra were recorded using Clariostar LVis plate from BMG Labtech. A calibration curve (Absorbance at 242 nm vs. TPA concentration) was obtained by measuring the absorbance of TPA (provided by Sigma Aldrich, purity 98%, used as received) aqueous solutions of NaOH 0.5 wt. % in milli-Q water of known concentrations, as presented in FIG. 31.

A linear fit of the calibration curve gives the following equation:

$$Absorbance @ 242 nm ( a . u . ) = 70.47 ( \frac{L}{g} ) * [ T P A ] ( \frac{g}{L} ) \qquad Equation 5$$

In what follows we use this calibration curve to convert the absorbance signal into the TPA concentration as if only TPA was produced. This method is called here determination of reaction yield by determination of TPA equivalent.

### Determination of TPA Equivalent Reaction Yield at 24 h for PET Materials

From the calibration curve presented in FIG. 31, it is possible to obtain the concentration of TPA after depolymerization reaction and calculate the corresponding reaction yield at 24 h (on average 23 hr). For an enzymatic depolymerization assay, around 5 mg (between 4.8 and 5.3 mg) (Sartorius CP224S, precision 0.1 mg) of milled material was weighted in a 2 mL Eppendorf vial. 1 mL of potassium phosphate buffer 1M (pH 8), prepared from potassium phosphate monobasic (H₂KPO₄) and potassium phosphate dibasic (HK₂PO₄) (Sigma Aldrich), was added in the Eppendorf vial. The Eppendorf vial was then cooled to 0° C. in ice.

Depolymerization assays were carried out using PET depolymerase LCC ICCG. The wild-type DNA sequence was obtained from GenBank (accession number: AEV21261) and mutated to make the ICCG variant as previously reported (Tournier et al., 2020). The amino acid sequence of interest, i.e., MSNPYQRGPNPTRSALTADGPFSVATYTVSRLSVSGFGGGVIYYPTGTSLTFGGI AMSPGYTADASSLAWLGRRLASHGFVVLVINTNSRFDGPDSRASQLSAALNYLR TSSPSAVRARLDANRLAVAGHSMGGGGTLRIAEQNPSLKAAVPLTPWHTDKTF NTSVPVLIVGAEADTVAPVSQHAIPFYQNLPSTTPKVYVELCNASHIAPNSNNAA ISVYTISWMKLWVDNDTRYRQFLCNVNDPALCDFRTNNRHCQ, was cloned to make an N-terminus His-tagged SUMO fusion construct, expressed in E. coli BL21 cells, and purified by immobilized metal affinity chromatography as follows: The lysate was suspended in 20 mM Tris pH 8, 300 mM NaCl and was bound to a Ni-Sepharose column. The column was washed with 50 mM imidazole, 20 mM Tris pH 8, 300 mM NaCl) and the His-tagged protein was eluted with 300 mM imidazole, 20 mM Tris pH 8, 300 mM NaCl. The His-SUMO tag was cleaved with Ulp1 (1:100 w Ulp1/w His-tagged protein) overnight in 20 mM Tris pH 8, 300 mM NaCl and was removed by passing through a Ni-Sepharose column, where the His-SUMO tag is adsorbed, and LCC passes through. LCC was concentrated and dialyzed against 50 mM Na-phosphate buffer, pH 8, 100 mM NaCl, and 10% glycerol. Aliquots of LCC were flash-frozen and stored at −80° C.

In each vial, a volume of 9.43*(m/5) μL of LCC ICCG stock solution was added to reach a final concentration of 2 mg of enzyme per g of polyester in the vial, were is the weighted mass in mg of plastic waste.

Then, the Eppendorf vial was closed, sealed with PTFE film to prevent/minimize evaporation, and incubated in an Eppendorf Thermomixer at a 65° C. and shaken at 1200 rpm during 24 hours.

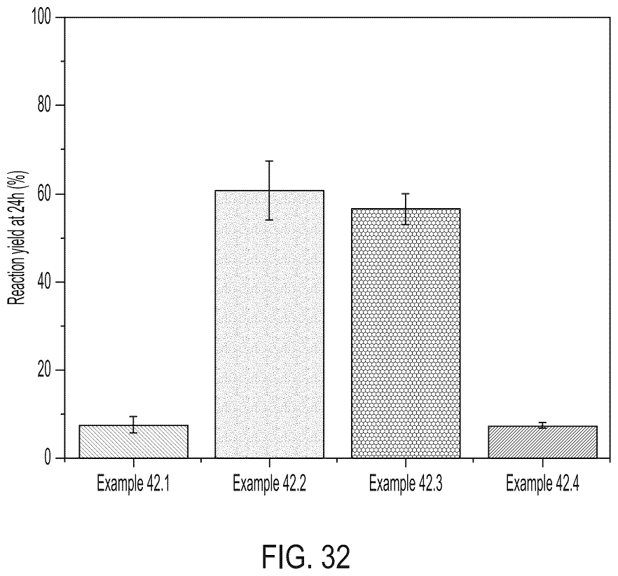

At 24 h of reaction, the PTFE film was removed and a 2 μL aliquot was taken from the reaction medium and was diluted (if required) by a factor of 5, 10 or 20 in NaOH 0.5 wt. % solution to ensure that the absorbance at 242 nm was in the linear range of TPA calibration curve presented in FIG. 5. The UV-Visible absorbance spectrum was recorded between 220 nm and 800 nm using a Clariostar LVis plate (BMG Labtech). The reaction yield was calculated as the concentration of TPA equivalents produced at 24 h in reference to the maximum TPA concentration (g/L) corresponding to 100% reaction yield, as follows:

$$Reaction yield ( \% ) = \frac{( \frac{{A}_{242 nm} {f}_{d}}{70.47} )}{\frac{m}{V} ( \frac{{M}_{A}}{{M}_{R U}} )} * 100 \qquad Equation 6$$

where A_{242 nm} is the absorbance of 2 μL (diluted) aliquot, f_{d} is the dilution factor of the aliquot, m is the weighted mass of plastic material waste of the assay (in g), V is the reaction volume (in L) M_{A} is the molecular weight of TPA, and M_{RU} is the molecular weight of the repeating unit of PET.
The reaction yield at 24 h (approximately 23 hours) was obtained by averaging the reaction yield of triplicate samples, i.e. 3 aliquots taken from 3 different vials in the same conditions, with error bars corresponding to the standard deviation of the three measurements. Enzymatic depolymerization reaction yield at 24 h (approximately 23 hours) of milled and sieved samples described in Example 42.1, Example 42.2, Example 42.3, and Example 42.4 are shown in FIG. 32.

The results show that milling a biaxially oriented polyester PET film (Example 42.3) is particularly helpful to achieve a significantly higher enzymatic depolymerization yield (23 h) compared to milling non-oriented PET samples (Example 42.1 and Example 42.4) under essentially identical conditions.

Furthermore, the example shows that the enzymatic depolymerization yield of just milled biaxially oriented PET films (Example 42.3) is, within the experimental error, as high as that obtained by a classical amorphisation procedure followed by milling the sample under essentially identical conditions (Example 42.2). Therefore, milling a stress or flow-induced oriented/crystallized polymeric material is particularly advantageous to obtain high enzymatic depolymerization yields without the need of additional energy-intensive steps such as extrusion prior to enzymatic depolymerization reaction.

### Example 43

### Simulating Grade C

A mixture of PET and HDPE was generated to simulate a Grade C bottle bale. This mock blend was created by combining 85% cleaned and sorted PET flakes (PQ Recycling) with 15% HDPE flakes (Inspired Plastics) in a bag. Materials were shaken until PET flakes and HDPE flakes were well mixed. The mass ratio of PET to HDPE flakes was based on the published industrial Grade C PET bales standard.

### Extrusion Procedure

Simulated Grade C material was flood fed into a single screw extruder (Filabot EX2). The extrusion was done at 50% screw speed and a barrel temperature of 280° C. Extrudate was immediately cooled in a water bath. Cooled extrudate was cut with a scissor to a length size of 1-2 cm

### Milling/Sieving Procedure

All samples were milled and processed with essentially identical conditions. Material was fed into a centrifugal mill (Retsch ZM200) using a 0.25 mm conidur hole short ring sieve (10 mm diameter) at 18,000 RPM with a vacuum cyclone collection system. The feed rate of pellets into the mill was controlled using a Single Screw Gravimetric Feeder (Movacolor MCBalance) at 0.25 kg/hr.

Each of the milled samples were fractionated by sieving using an analytical sieve shaker (Retsch, AS200) operating at an amplitude of about 1 mm/“g” (30 minutes of total shaking). Fractionation was performed using stainless steel test sieves (Retsch) with a diameter of 200 mm and mesh sizes of: 500 μm, 400 μm, 300 μm, 200 μm, 100 μm, and 50 μm.

### Sink Float Operation

Pre-processed 200-300 m Grade C material was subjected to a sink-float operation where the material was placed in a 2-liter beaker filled with 1 liter of water. The material was stirred at 500 RPM (Achiever 5000, OHaus) until thorough mixing of the material and water was observed. Once mixing ceased, the material was allowed to settle for 15 minutes. The float and sunk fractions were collected and vacuum filtered to remove excess water.

### Enzymatic Depolymerization

Three samples were generated for enzymatic depolymerization—1) PET/HDPE blend milled only; 2) PET/HDPE blend milled followed by sink-float operation; 3) PET/HDPE blend extruded together, then milled followed by sink-float. The recovered fractions of each sample were enzymatically depolymerized in bioreactors following the same method outlined in Example 28, using the volume of sodium hydroxide dosed to monitor reaction extent.

The reactor containing 10 g of the milled material dosed 13.97 mL of 6M NaOH after 48 hours, corresponding to a reaction completeness of 80.6%. The reactor containing 10 g of milled sink float material dosed 15.69 mL of 6M NaOH after 48 hours, corresponding to a reaction completeness of 90.5%. Finally, the reactor containing 10 g of extruded sink float material dosed 13.58 mL of 6M NaOH after 48 hours, corresponding to a reaction completeness of 78.3%. These reactions show a 10% improvement in depolymerization by adding a sink-float operation to the milled PET/HDPE blend. Additional details of the reaction method are described below.

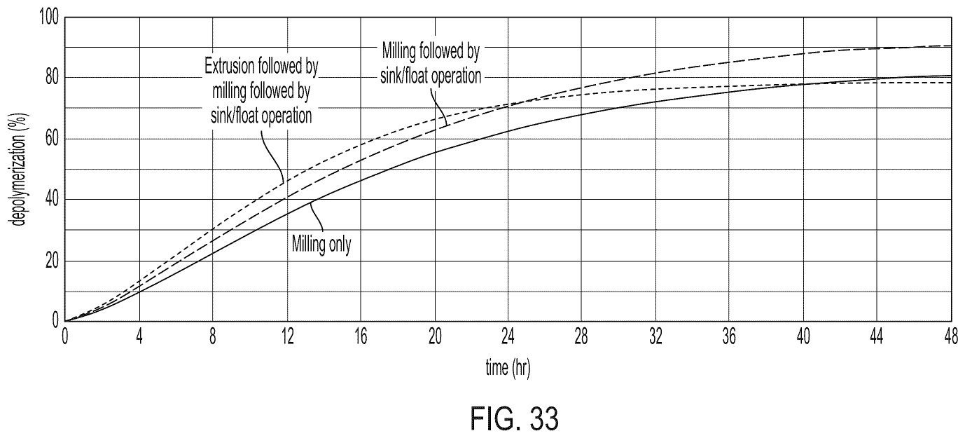

Three samples were generated for enzymatic depolymerization—1) PET/HDPE blend milled only; 2) PET/HDPE blend milled followed by sink-float operation; 3) PET/HDPE blend extruded together, then milled followed by sink-float. The results are shown in FIG. 33.

The recovered fractions of each sample were next enzymatically depolymerized in bioreactors using Novozyme HiC 51032 (Lot L01332211, purchased from Strem Chemicals). The reaction was operated at 65 C, 10% solid loading (10 g in 90 mL), 100 millimolar potassium phosphate buffer at pH 8.0, with 5 mL of enzyme solution as received. The pH of the reaction was controlled using a Raspberry Pi controlled system which doses 6 molar sodium hydroxide to maintain pH at 8.0.

The reaction progression and kinetics can be tracked by the volume of sodium hydroxide dosed throughout the reaction. This relationship between dosed sodium hydroxide and extent of reaction is described by the following Equation 3. For 10 grams of PET, the theoretical yield of terephthalic acid is 8.64 grams. The reactor containing 10 g of the milled material dosed 13.97 mL of 6M NaOH after 48 hours, corresponding to a reaction completeness of 80.6%. The reactor containing 10 g of milled sink float material dosed 15.69 mL of 6M NaOH after 48 hours, corresponding to a reaction completeness of 90.5%. Finally, the reactor containing 10 g of extruded sink float material dosed 13.58 mL of 6M NaOH after 48 hours, corresponding to a reaction completeness of 78.3%. These reactions show a 10% improvement in depolymerization by adding a sink-float operation to the milled PET/HDPE blend.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-44. (canceled)

45. A method of processing a crystallizable polymer or copolymer and/or a PC/IPM containing the crystallizable polymer or copolymer, comprising:
milling the crystallizable polymer or copolymer to reduce a percentage of crystallinity of the crystallizable polymer or copolymer in a mill,
at an apparent mass flow rate of at least 0.01 kg of polymer per hour within the mill, and/or with a mean residence time of the polymer within the mill of less than 30 minutes, at a mill temperature no more than T_{g}+100° C., wherein T_{g} is the glass transition temperature of the crystallizable polymer or copolymer.

46. The method of claim 45, wherein the milled crystallizable polymer or copolymer has a percentage of crystallinity that is at least 2% less than the percentage of crystallinity of the crystallizable polymer or copolymer before the milling.

47. The method of claim 45, wherein the milled crystallizable polymer or copolymer is in the form of a plurality of polymeric particles.

48. The method of claim 45, further comprising degrading the milled crystallizable polymer or copolymer with an enzyme.

49. The method of claim 45, wherein the crystallizable polymer or copolymer has a percentage of crystallinity of greater than or equal to 1% and less than or equal to 90%.

50. The method of claim 45, wherein the milled crystallizable polymer or copolymer has an average percentage of crystallinity of greater than or equal to 1% and less than or equal to 50%.

51. The method of claim 45, wherein the milled crystallizable polymer or copolymer comprises a plurality of polymeric particles having an average particle size of greater than or equal to 5 μm and less than or equal to 5 mm.

52. The method of claim 45, further comprising adjusting a rate of temperature increase during milling such that the rate is greater than or equal to 0.1° C. per minute and less than or equal to 50° C. per minute.

53. The method of claim 45, wherein the mill has a milling power of at least 0.01 hp and up to 3000 hp.

54. The method of claim 45, wherein the crystallizable polymer or copolymer has a glass transition temperature of greater than or equal to −150° C. and less than or equal to 250° C.

55. The method of claim 45, further comprises separating each of the plurality of polymeric particles by composition after milling the crystallizable polymer or copolymer.
